(19) United States
(12) Patent Application Publication
(10) Pub. No.: US 2024/0175863 A1
(43) Pub. Date: May 30, 2024

(54) COMPOSITIONS AND METHODS FOR MONITORING ENPP1 ACTIVITY

(71) Applicant: The Regents of University of Californa, Oaklan, CA (US)

(72) Inventor: Arjun Deb, Los Angeles, CA (US)

(21) Appl. No.: 18/278,995

(22) PCT Filed: Mar. 3, 2022
(86) PCT No.: PCT/US22/18633

§ 371 Date: Aug. 25, 2023

Related U.S. Application Data
(60) Provisional application No. 63/156,739, filed on Mar. 4, 2021.

(51) Int. Cl. G01N 33/50; A61K 31/352; A61K 31/7072; A61K 45/06; A61P 9/10

(52) U.S. Cl. CPC G01N 33/5061; A61K 31/352; A61K 31/7072; A61K 45/06; A61P 9/10; G01N 33/502; G01N 2333/916; G01N 2800/325

(57) ABSTRACT

Provided herein are methods and compositions related to monitoring ENPP1 activity and the treatment of cardiac injury using pyrimidine nucleotides.

### RELATED APPLICATIONS

This application claims the benefit of priority to U.S. Provisional Patent Application Ser. No. 63/156,739, filed Mar. 4, 2021, the contents of which are hereby incorporated herein by reference in their entirety.

### GOVERNMENT SUPPORT

This invention was made with government support under Grant Numbers HL137241, AR075867, HL149687, and HL149658, awarded by the National Institutes of Health, and Grant Numbers W81XWH-17-1-0464 and W81XWH-20-1-0238, awarded by the Department of Defense. The government has certain rights in the invention.

### BACKGROUND

The heart possesses a poor ability to regenerate dead cardiac muscle after acute ischemic injury and lost heart muscle is replaced by non-contractile scar tissue. Scar tissue increases the hemodynamic burden on the remaining cardiac muscle and over time, the ventricle fails leading to a vicious cycle of ventricular dilatation, worsening fibrosis and progressive decline in cardiac function. More than 700,000 patients are annually diagnosed with heart failure and more than 40% of cases of heart failure worldwide result from heart attacks or myocardial infarction. Heart failure is a major cause of death worldwide. Braunwald's Heart Disease, 11.sup.th ed. (2015). It has an estimated prevalence of 38 million patients worldwide, a number that is increasing with the ageing of the population. Braunwald, E. The War against Heart Failure. Lancet 385:812-824 (2015). The prognosis of heart failure is worse than that of most cancers. Thus, modulation of cardiac wound healing to redirect the cardiac injury response from a fibrotic to a reparative one with minimal adverse remodeling and decline in heart function remains a broad goal of cardiovascular therapeutics. There remains a long-felt and unmet need for novel cardiovascular therapeutics for the treatment of cardiac injury. Furthermore, new methods are needed to prevent and monitor cardiac injury.

### SUMMARY

Disclosed herein are compositions and methods related to monitoring ENPP1 activity. Such compositions and methods can be used, for example, to treat myocardial infarction, promote cardiac wound healing, enhance cardiac repair, inhibit ENPP1 activity, prevent heart failure, prevent cardiac cell death, prevent ectopic calcification of cardiac tissue, prevent scarring of cardiac tissue, prevent dilated cardiomyopathy, or prevent release of one or more pro-inflammatory molecules from cardiac myocytes in a subject. Accordingly, in certain embodiments, provided herein are methods of monitoring ENPP1 activity (e.g., determining a level of a pyrimidine nucleotide in the subject), treating cardiac injury in a subject (e.g., administering a pyrimidine nucleotide to a subject), and identifying a candidate ENPP1 inhibitor (e.g., contact a cell sample with a test agent and measuring a level of a pyrimidine nucleotide of the cell sample).

### BRIEF DESCRIPTION OF THE DRAWINGS

FIG. 1A shows qPCR demonstrating ENPP1 gene expression in the injured region of the heart compared to uninjured regions at 3, 7, 14 and 21 days after myocardial infarction (mean ±S.E.M. n=5, **p<0.01, *p<0.05).

FIG. 1B shows Western blotting and quantitative densitometry demonstrating ENPP1 protein level in injured and uninjured regions of the heart at 7 days following infarction (mean ±S.E.M. n=3, **p<0.01).

FIG. 1C shows ATP hydrolytic activity at various concentrations of the injured heart tissue homogenate compared to uninjured tissue homogenate 7 days after injury (mean ±S.E.M. n=3, **p<0.01, *p<0.05).

FIG. 1D shows a heatmap with gene expression patterns of ENPP1 (arrow) and other members of the ENPP and ectonucleotidase family in the injured versus uninjured regions of the heart (n=4/time point, Un:Uninjured & In:Injured tissue). (E) ATP hydrolytic activity at 7 days post MI in wild type mice and ENPP1asj/asj mutant mice (mean ±S.E.M. n=3, ** p<0.01, *p<0.05).

FIG. 1E shows ATP hydrolytic activity at 7 days post MI in wild type mice and ENPP1asj/asj mutant mice (mean ±S.E.M. n=3, **p<0.01, *p<0.05).

FIG. 1F shows hematoxylin & eosin stain and immunostaining for ENPP1 (green, arrows) in the uninjured regions at day 7 post MI.

FIG. 1G shows hematoxylin & eosin stain and immunostaining for ENPP1 (green, arrows) in the injured regions at day 7 post MI.

FIG. 1H shows immunostaining for ENPP1 and Vimentin in the uninjured and injured region at 7 days post MI (arrowheads indicate ENPP1 and Vimentin colocalization in merged image).

FIG. 1I shows immunostaining of ENNP1 expression in genetically labeled cardiac fibroblasts in Col1a2CreERT:R26R^{tdtomato} mice at 7 days following injury. (arrowheads indicate where ENPP1 expressing cells co-express the fibroblast tdTomato label, representative images, n=3).

FIG. 1J shows immunostaining of ENNP1 expression in genetically labeled cardiac fibroblasts in TCF21MCM:R26R^{tdtomato} mice at 7 days following injury. (arrowheads indicate where ENPP1 expressing cells co-express the fibroblast tdTomato label, representative images, n=3).

FIG. 1K shows single cell RNA-seq of non-myocytes at 7 days post MI demonstrating cell phenotypes in clusters and ENPP1 distribution (n=3).

FIG. 2A shows co-culture of rat ventricular cardiomyocytes (CMs, red) with Control or ENPP1 overexpressing cardiac fibroblasts (Control-CF, ENPP1-CF, green) in the presence or absence of added ATP (arrows show decrease in ENPP1-CF, when ATP is added).

FIG. 2B shows number of CMs and cardiac fibroblasts under these conditions following 48 hours of co-culture (mean ±S.E.M. n=4, **p<0.01, ns: not significant).

FIG. 2C shows control CFs or ENPP1 CFs (green) in the presence or absence of added ATP but without any co-culture with cardiomyocytes, and quantitation of cell numbers under these conditions after 48 hours of ATP/vehicle addition (mean ±S.E.M. n=3, ns: not significant).

FIG. 2D shows transfer of control or ENPP1+ATP myocyte conditioned medium (MCndM) to cardiac fibroblasts and photomicrographs 48 hours later demonstrating decreased number of CF treated with ENPP1+ATP MCndM (arrows).

FIG. 2E shows flow cytometry with Propidium iodide and Annexin V to demonstrate the fraction of dead (PI+) or apoptotic cells (Annexin V+, PI−) following treatment with ENPP1+ATP MCndM or control MCndM (mean ±S.E.M. n=7, **p<0.01).

FIG. 2F shows TUNEL and caspase staining of cardiac fibroblasts treated with vehicle MCndM or ENPP1+ATP MCndM.

FIG. 2G shows quantitation of TUNEL+ or Caspase 3+ cells (mean ±S.E.M. n=3, ** p<0.01).

FIG. 2H shows cardiac fibroblasts treated with Vehicle MCndM, PPi MCndM or AMP MCndM for 48 hours showing loss of cells with treatment with AMP MCndM (arrow).

FIG. 21 shows quantitation of dead cells with flow (mean ±S.E.M. n=7, **p<0.01).

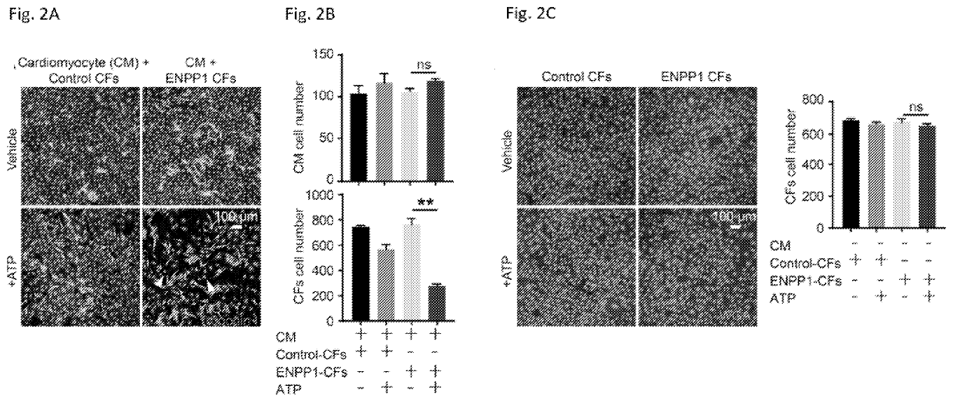

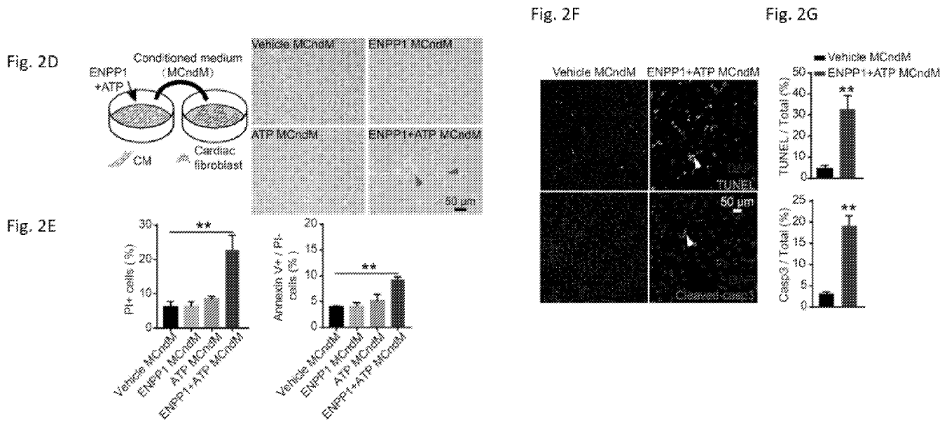

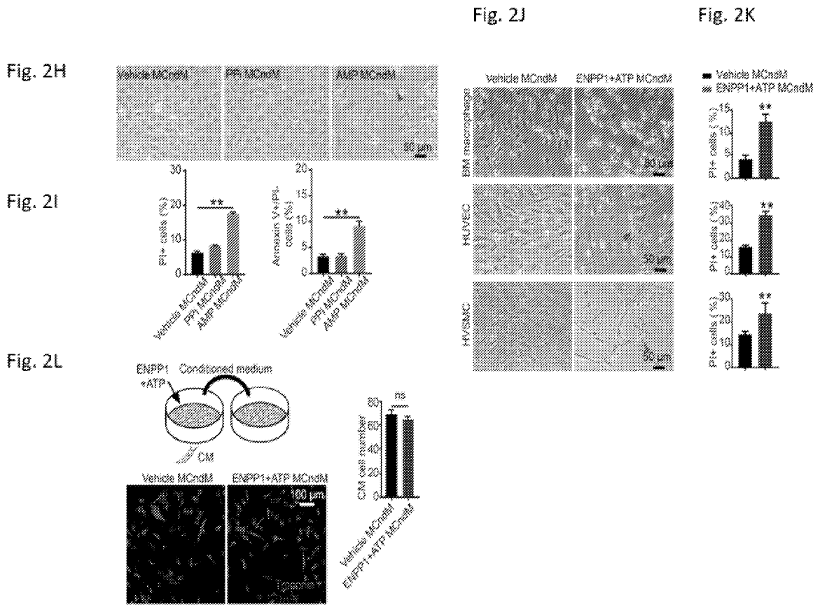

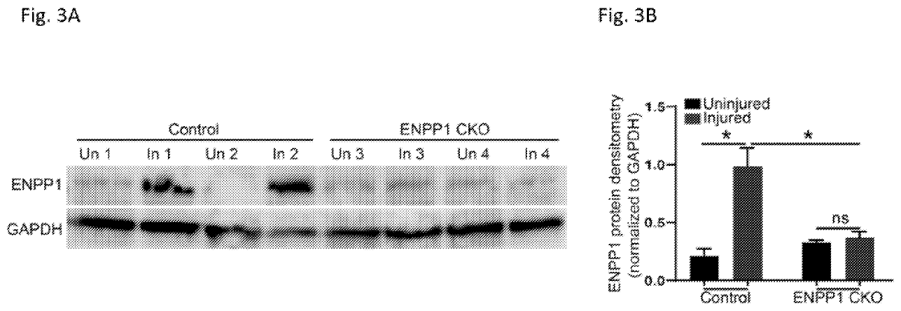

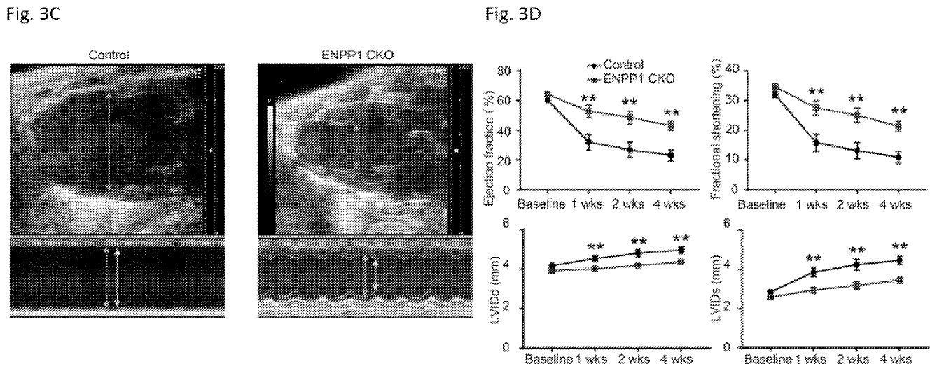

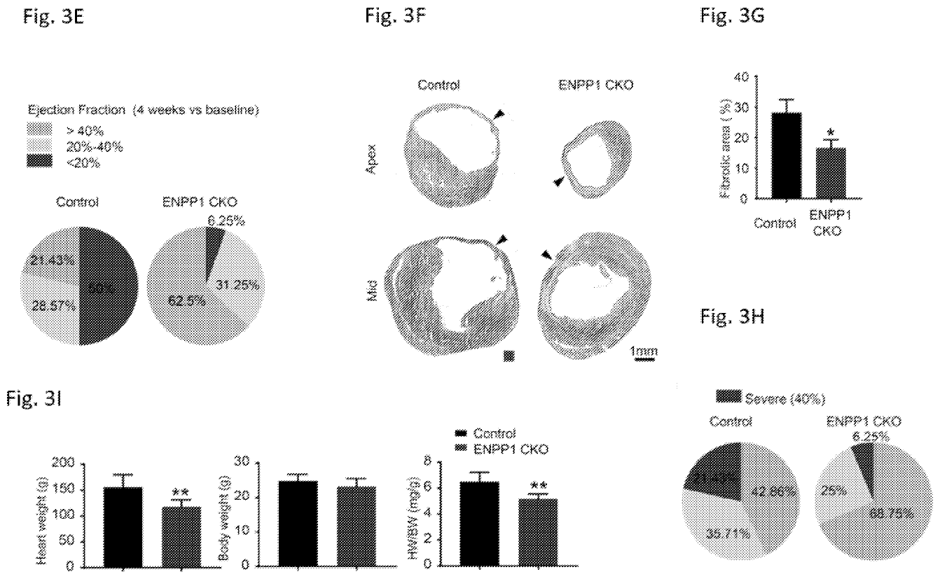

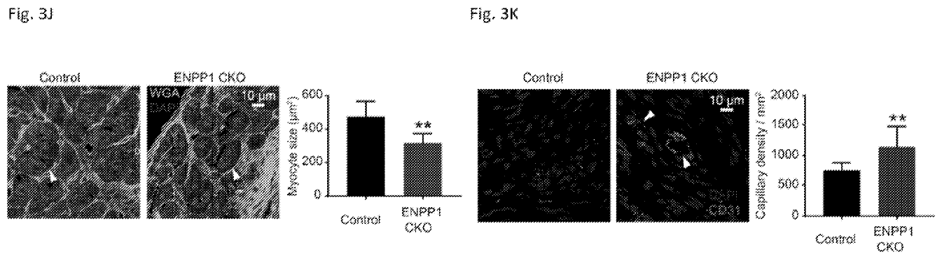

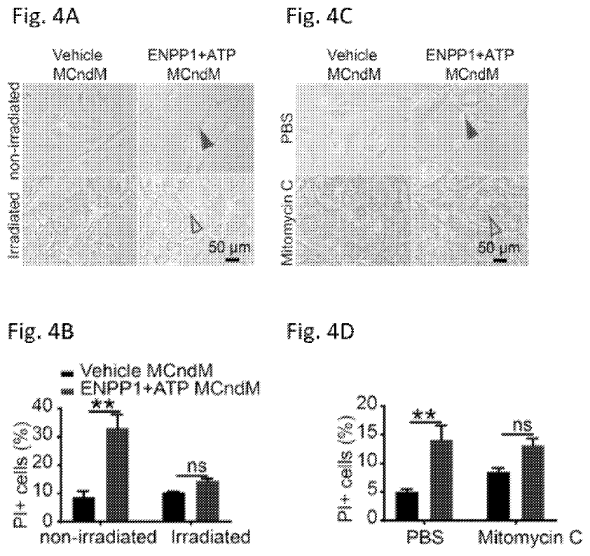

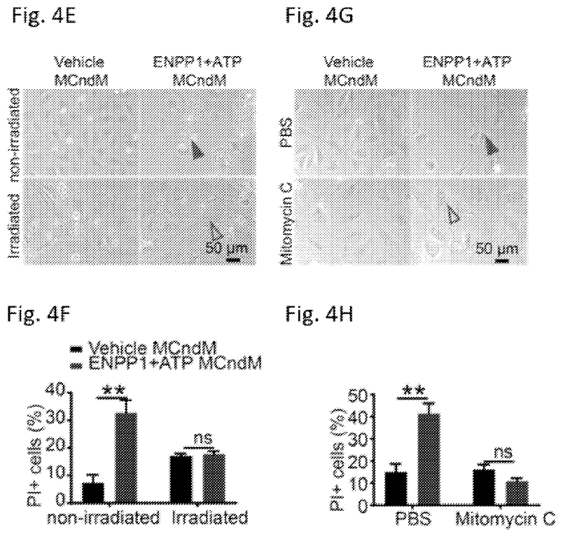

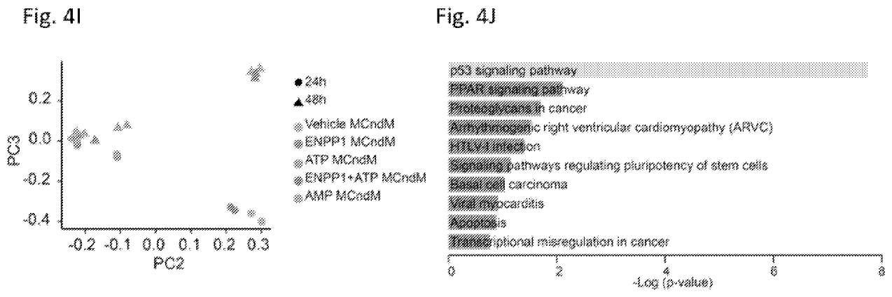

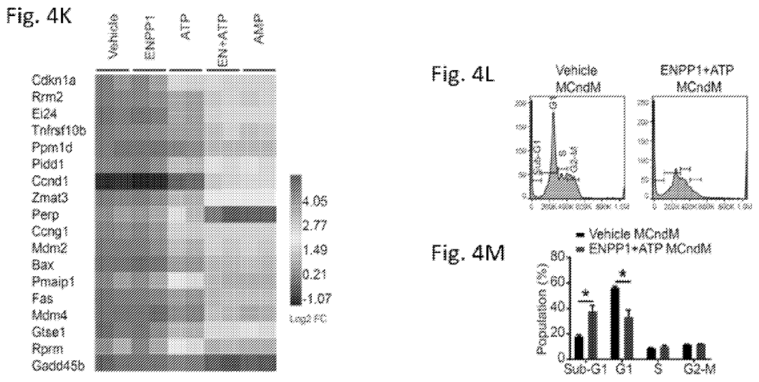

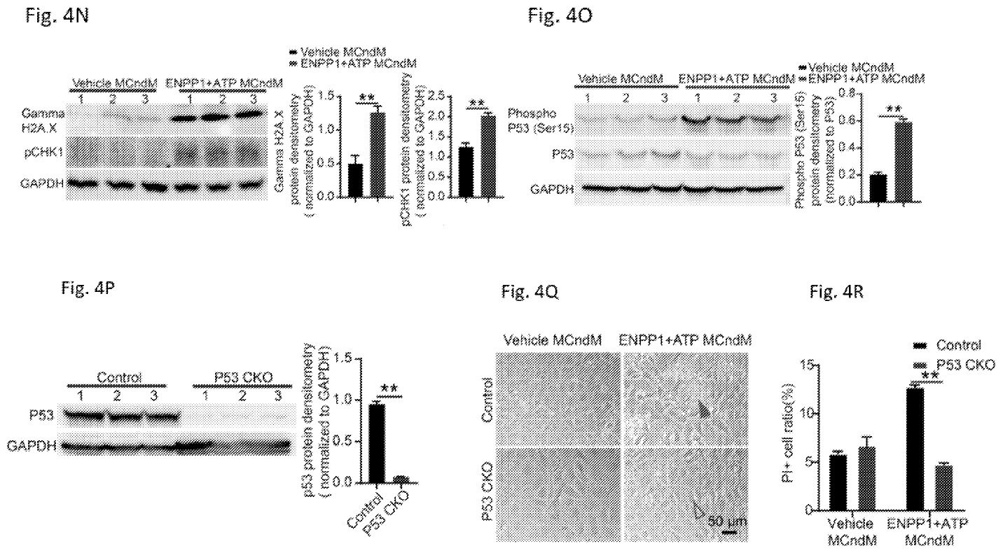

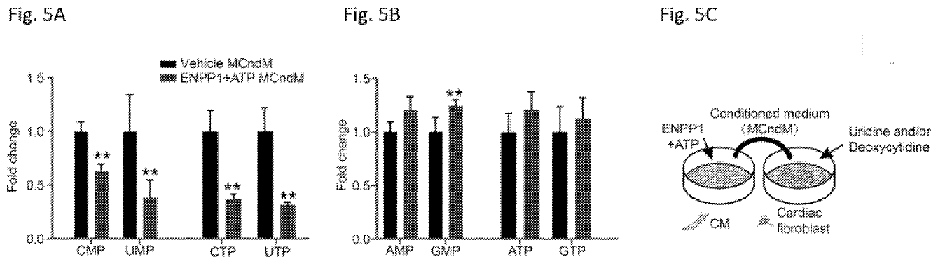

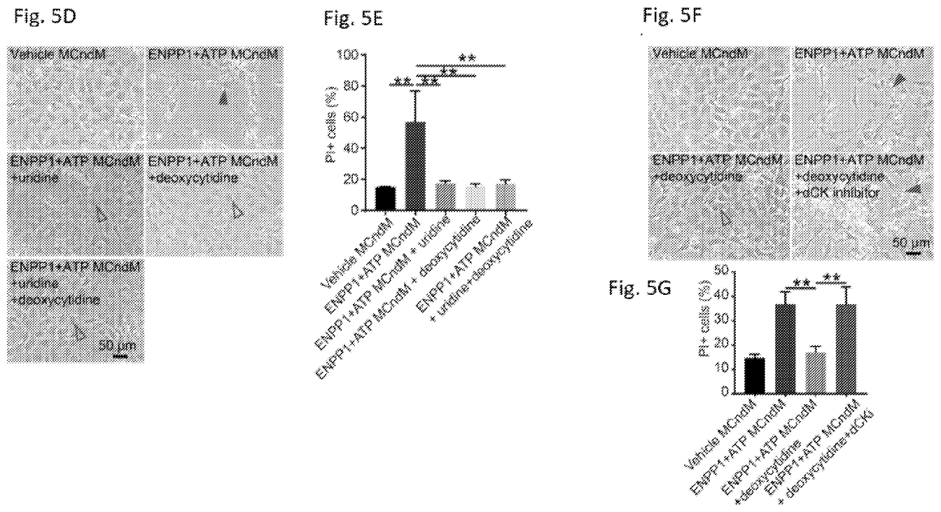

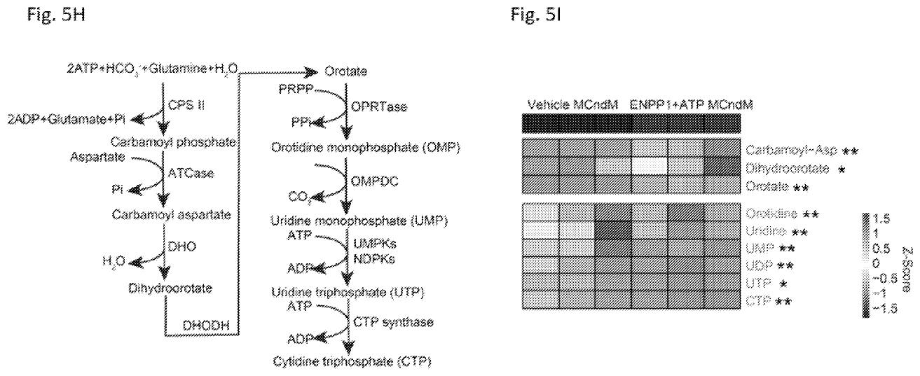

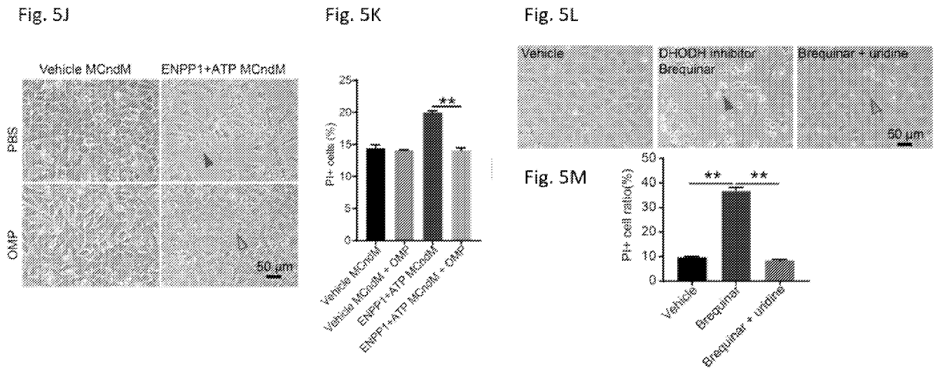

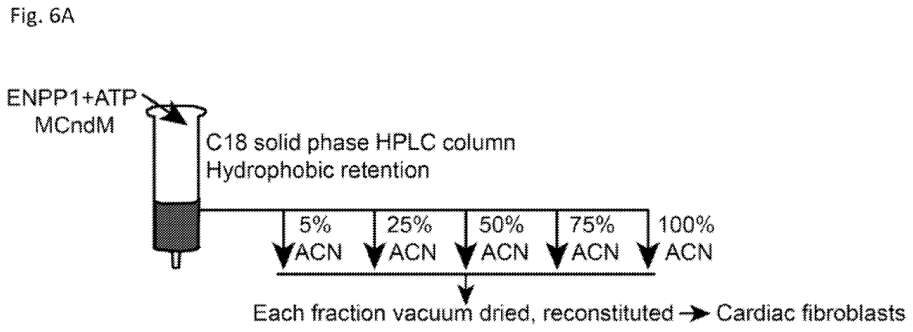

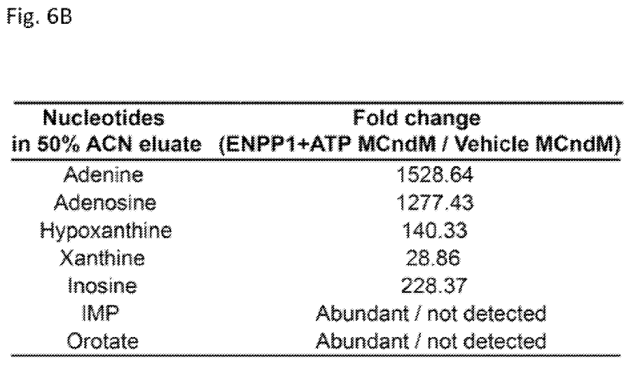

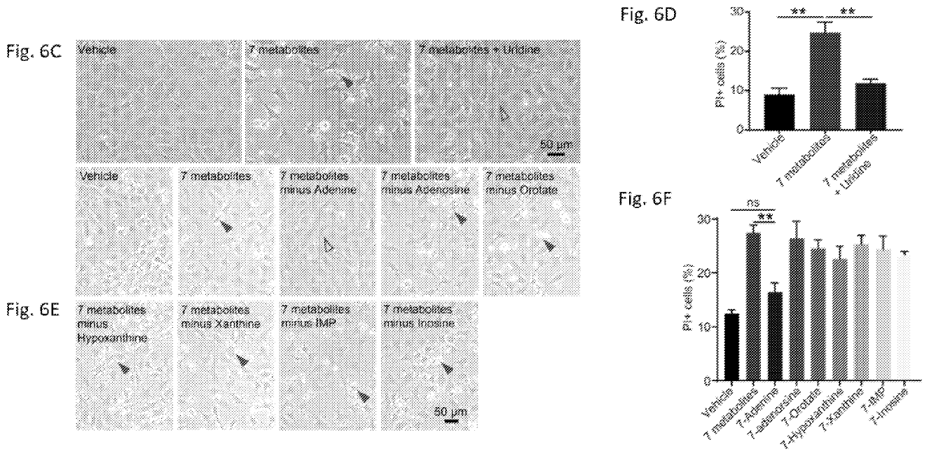

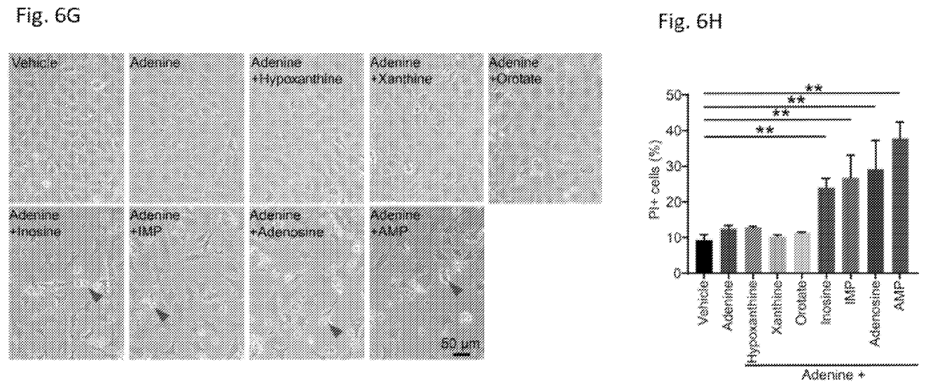

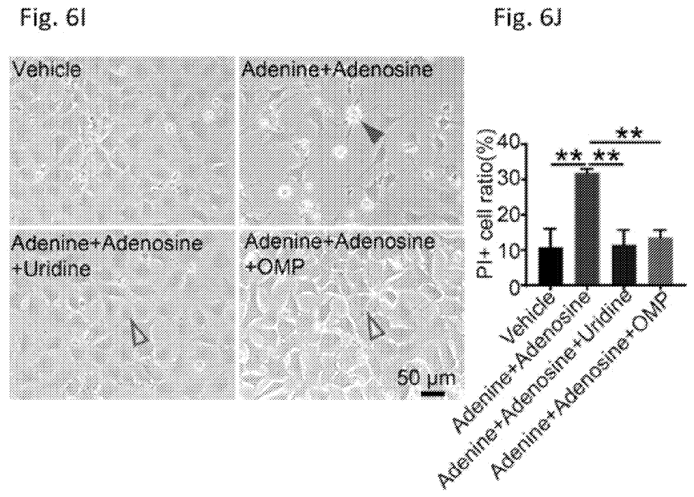

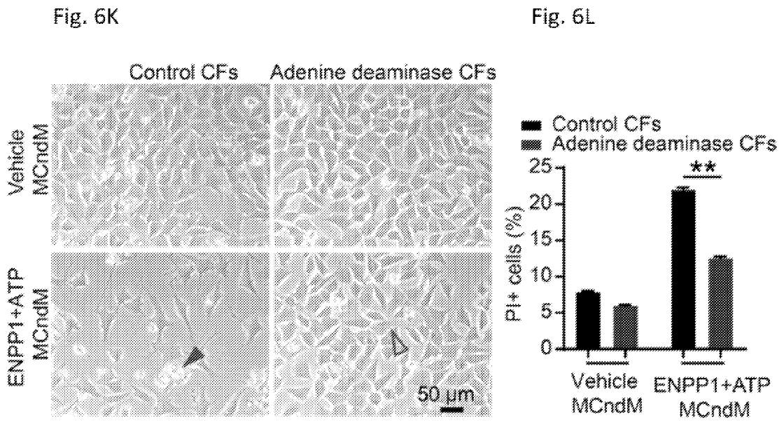

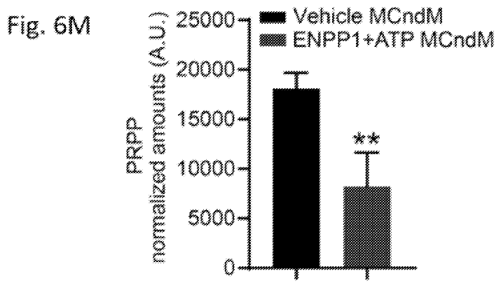

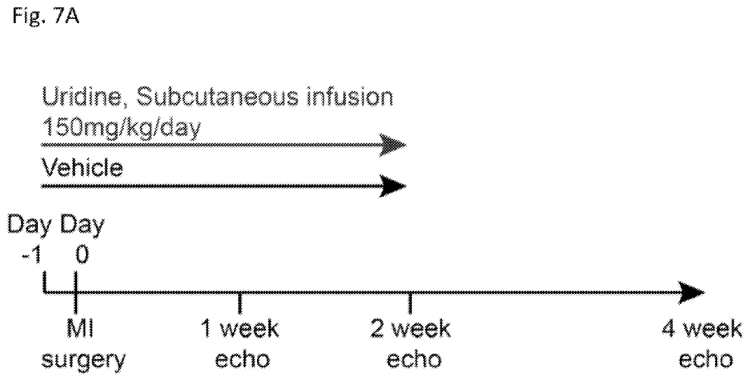

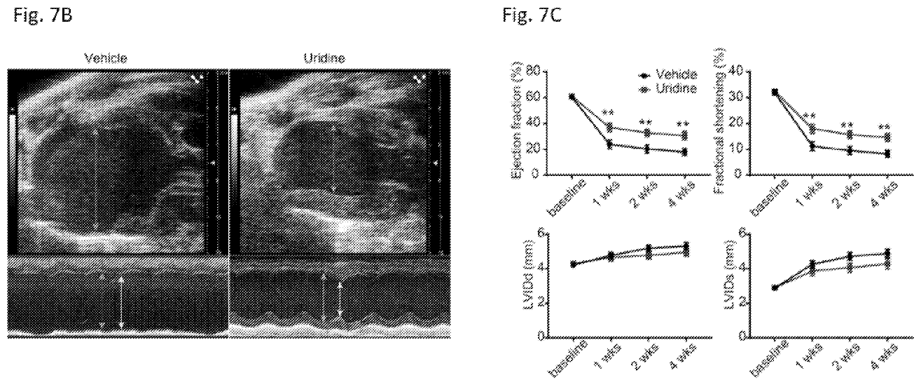

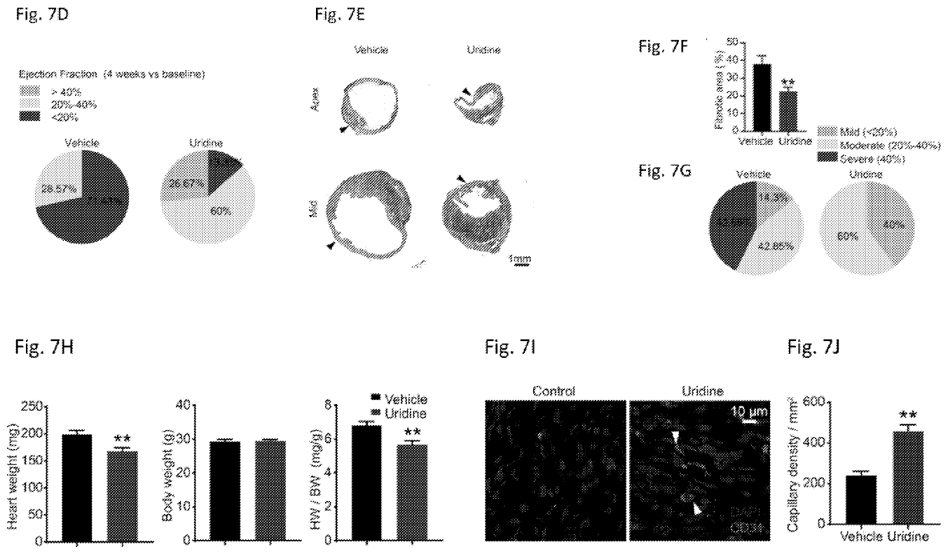

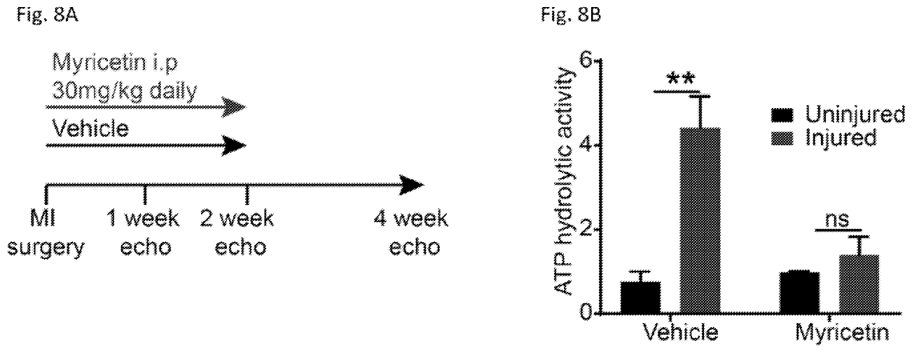

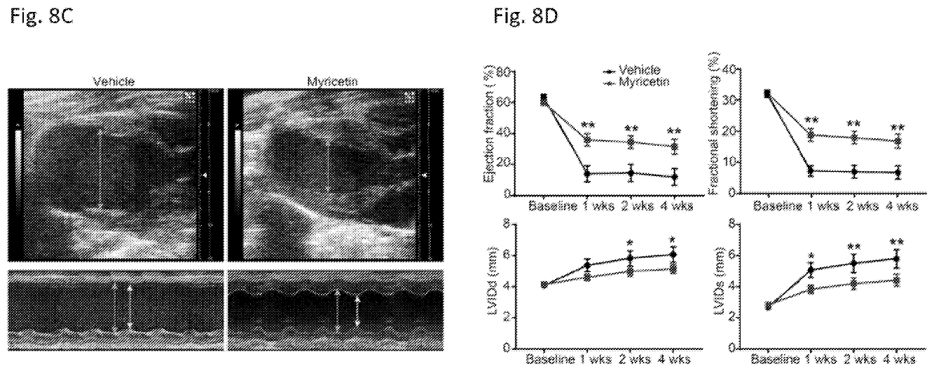

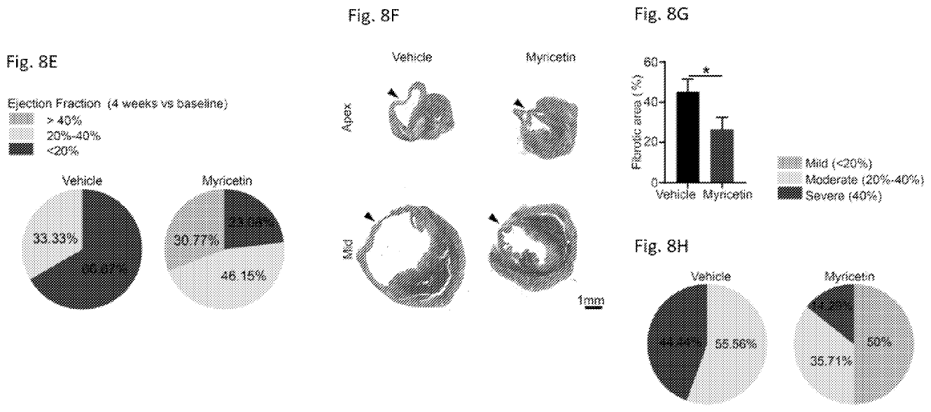

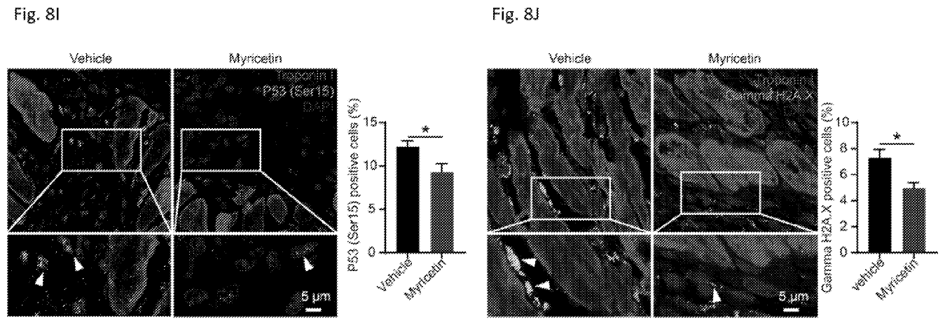

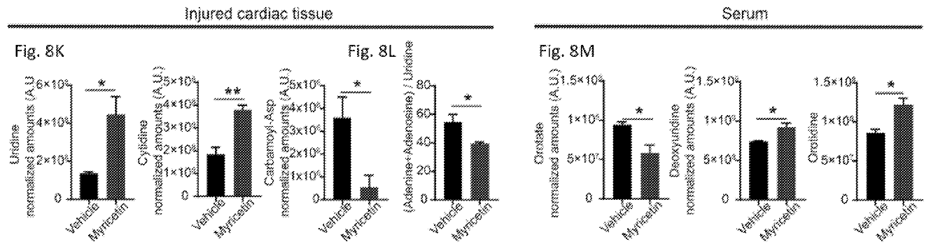

FIG. 2J shows treatment of macrophages, human endothelial (HUVEC) and human vascular smooth muscle cells (hVSMC) with vehicle MCndM or ENPP1+ATP MCndM.

FIG. 2K shows corresponding flow cytometry to determine cell death (mean ±S.E.M. n=3, **p<0.01).

FIG. 2L shows ENPP1+ATP MCndM does not cause cell death when added to myocytes (mean ±S.E.M. n=4).

FIG. 3A shows Western blotting demonstrating decreased ENPP1 expression in the hearts of ENPP1CKO animals at 7 days following cardiac injury.

FIG. 3B shows quantitation of ENPP1 protein expression post injury (mean ±S.E.M., *p<0.05).

FIG. 3C shows B mode and M mode echocardiogram demonstrating better contractile function with decreased chamber dilatation at 4 weeks following cardiac injury (green arrow: diastole; yellow arrow: systole).

FIG. 3D shows ejection fraction and Fractional shortening as well as left ventricular chamber size (LVID) in systole (s) and diastole (d) over 4 weeks after cardiac injury in control and ENPP1CKO animals (mean ±S.E.M. n=14 in control and n=16 in ENPP1CKO animals, **p<0.01).

FIG. 3E shows a pie chart demonstrating fraction of animals with mild, moderate and severe reductions in EF.

FIG. 3F shows masson trichrome staining demonstrating scar size (blue) measured at the apex and mid ventricle in control and ENPP1CKO animals.

FIG. 3G shows quantitation of differences in scar size as a fraction of the left ventricular surface area (mean ±S.E.M. n=14 in control and n=16 in ENPP1CKO animals, **p<0.01).

FIG. 3H shows a pie chart demonstrating fraction of animals demonstrating mild, moderate and severe fibrosis.

FIG. 3I shows heart weight (HW), body weight (BW) and HW/BW ratios measured at 4 weeks following cardiac injury and (mean ±S.E.M. n=14 in control and n=16 in ENPP1CKO animals, **p<0.01).

FIG. 3J shows cardiac troponin T immunostaining to determine myocyte surface area (arrows) at the border zone of control and ENPP1CKO animals and quantitation of myocyte surface area (mean ±S.E.M. n=14 in control and n=16 in ENPP1CKO animals, **p<0.01).

FIG. 3K shows number of capillaries (identified by CD31 staining, arrows) in ENPP1CKO and control animals at 4 weeks after heart injury and quantitation of capillary density (mean ±S.E.M. n=14 in control and n=16 in ENPP1 CKO animals, **p<0.01).

FIG. 4A shows irradiated or non-irradiated cardiac fibroblasts (CF) were treated with vehicle myocyte conditioned medium (MCndM) or ENPP1+ATP MCndM and photomicrographs 48 hours later shows cell death in non-irradiated CF treated with ENPP1+ATP MCndM (arrows) but not in irradiated CF (unfilled arrow).

FIG. 4B shows quantitation of cell death under these conditions (mean ±S.E.M. n=3, **p<0.01).

FIG. 4C shows PBS or mitomycin C treated CF were subjected to treatment with vehicle MCndM or ENPP1+ATP MCndM and photomicrographs taken 48 hours later shows rescue of cell death with mitomycin (filled and unfilled arrows).

FIG. 4D shows flow cytometry to quantitate cell death (mean ±S.E.M. n=5, **p<0.01).

FIG. 4E shows mouse embryonic fibroblasts (mEF) treated with Vehicle MCnDM or ENPP1+ATP MCndM following irradiation and photomicrographs 48 hours later demonstrate rescue of cell death with irradiation or mitomycin C (filled and unfilled arrows).

FIG. 4F shows flow cytometry to quantitate cell death of mEF following irradiation (mean ±S.E.M. n=3, **p<0.01).

FIG. 4G shows mouse embryonic fibroblasts (mEF) treated with Vehicle MCnDM or ENPP1+ATP MCndM following mitomycin C and photomicrographs 48 hours later demonstrate rescue of cell death with irradiation or mitomycin C (filled and unfilled arrows).

FIG. 4H shows flow cytometry to quantitate cell death of mEF following mitomycin treatment (mean ±S.E.M. n=3, **p<0.01).

FIG. 4I shows principal component analysis of gene expression changes in cardiac fibroblasts treated with ENPP1+ATPMCndM, AMP MCndM or control MCndM, at 24 and 48 hours (n=2).

FIG. 4J shows gene ontology analysis of main pathways differentially expressed in cardiac fibroblasts following treatment with ENPP1+ATP MCndM.

FIG. 4K shows a heat map demonstrating expression of principal apoptotic genes in p53 signaling pathway that are differentially expressed (p<0.05) in cardiac fibroblasts treated with ENPP1+ATP MCndM.

FIG. 4L shows cell cycle analysis demonstrating Gl/S phase arrest in cardiac fibroblasts treated with vehicle MCndM or ENPP1+ATP MCndM.

FIG. 4M shows quantitation of fraction of cells in different phases of cell cycle (mean S.E.M. n=3, *p<0.05).

FIG. 4N shows a Western blot demonstrating expression of gamma H2A.X and pCHK-1 in cardiac fibroblasts treated with ENPP1+ATP MCndM (n=3, *p<0.05). (0) Western blot and densitometry demonstrating Ser15 phosphorylation in p53 in cardiac fibroblasts treated with ENPP1+ ATP MCndM (mean ±S.E.M. n=3, **p<0.01).

FIG. 4O shows a Western blot and densitometry demonstrating Ser15 phosphorylation in p53 in cardiac fibroblasts treated with ENPP1+ ATP MCndM (mean ±S.E.M. n=3, **p<0.01).

FIG. 4P shows p53 protein levels in wild type and p53CK0 cardiac fibroblasts.

FIG. 4Q shows photomicrographs of wild type or p53CK0 cardiac fibroblasts treated with vehicle MCndM or ENPP1 MCndM demonstrating rescue of cell death in the p53CK0 cardiac fibroblasts (filled and unfilled arrows).

FIG. 4R shows quantitation of cell death under these conditions (mean ±S.E.M. n=3, **p<0.01).

FIG. 5A shows LC/MS-MS demonstrating decreased levels of intracellular pyrimidine nucleotides.

FIG. 5B shows LC/MS-MS demonstrating unchanged levels of intracellular purine nucleotides.

FIG. 5C shows cardiac fibroblasts treated with vehicle MCndM or ENPP1+ATP MCndM in the presence of uridine, deoxycytidine or both.

FIG. 5D shows cell death (arrows) in cardiac fibroblasts treated with ENPP1+ATP MCndM but rescue of cell death (unfilled arrows) following addition of uridine, deoxycytidine or both.

FIG. 5E shows flow cytometry to demonstrate effects on cell death following addition of uridine or deoxycytidine to cardiac fibroblasts treated with ENPP1+ATP MCndM (mean ±S.E.M. n=6, **p<0.01).

FIG. 5F shows the effect of adding deoxycytidine and deoxycytidine kinase inhibitor (dCKi) to cardiac fibroblasts treated with ENPP1+ATP MCndM demonstrates loss of rescue of deoxycytidine in the presence of dCKi (unfilled and filled arrows).

FIG. 5G shows quantitation of cell death (mean ±S.E.M. n=5, **p<0.01).

FIG. 5H shows outline of critical steps of pyrimidine biosynthesis.

FIG. 5I shows heat map demonstrating differential expression of metabolites in pyrimidine biosynthetic pathway between CF treated with vehicle MCndM or ENPPl+CF MCndM (n=3, **p<0.01 for all metabolites shown).

FIG. 5J shows rescue of cell death following addition of orotidine monophosphate (01VIP) to cardiac fibroblasts treated with ENPP1+ATP MCndM (filled and unfilled arrows) and (K) quantitation of cell death (mean ±S.E.M. n=4, **p<0.01).

FIG. 5K shows quantitation of cell death (mean ±S.E.M. n=4, **p<0.01).

FIG. 5L shows effect on cell death following addition of DHODH inhibitor brequinar (filled arrows) to disrupt pyrimidine biosynthesis and rescue with uridine (unfilled arrows).

FIG. 5M shows flow cytometry to determine effects of brequinar on cell death and rescue by uridine (mean ±S.E.M. n=5, **p<0.01).

FIG. 6A shows the effects of ENPP1+ATM MCndM eluted from a C18 column by various concentrations of acetonitrile on cell death (arrows) of cardiac fibroblasts (mean ±S.E.M. n=3, **p<0.01).

FIG. 6B shows nucleosides/bases that were enriched in the 50% ACN elutes of ENPP1+ATP MCndM versus vehicle MCndM.

FIG. 6C shows effect of 7 metabolites on cell death (filled arrows) of cardiac fibroblasts and its rescue (unfilled arrows) by uridine.

FIG. 6D shows quantitation of cell death under these conditions (mean ±S.E.M. n=9, **p<0.01).

FIG. 6E shows effects on cell death (filled arrows) of cardiac fibroblasts treated with 7 compounds together and following subtraction of each one from the combined solution demonstrates absence of cell death when adenine (unfilled arrow) is removed.

FIG. 6F shows quantitation of cell death to demonstrate that exclusion of adenine leads to rescue of cell death (mean ±S.E.M. n=3, **p<0.01).

FIG. 6G shows effects of cell death (filled arrows) following addition of adenine alone or adenine combined with specific purine nucleosides or orotate.

FIG. 6H) shows quantitation of cell death under these conditions (mean ±S.E.M. n=3, **p<0.01).

FIG. 6I shows effect of OMP or uridine in rescuing cell death following addition of adenine and adenosine to cardiac fibroblasts (filled and unfilled arrows).

FIG. 6J shows quantitation of cell death (mean ±S.E.M. n=6, **p<0.01).

FIG. 6K shows cardiac fibroblasts over-expressing yeast adenine deaminase treated with vehicle MCndM or ENPP1+ATP MCndM and photomicrographs show decreased cell death of cardiac fibroblasts overexpressing adenine deaminase (filled and unfilled arrows).

FIG. 6L shows reduction of cell death in adenine deaminase expressing cardiac fibroblasts (mean ±S.E.M. n=3, **p<0.01).

FIG. 6M shows decreased PRPP levels in cardiac fibroblasts treated with ENPP1+ATP MCndM (mean ±S.E.M. n=4, **p<0.01).

FIG. 7A shows schematic of continuous uridine administration by a subcutaneous pump starting one day prior to injury and continuing for 14 days.

FIG. 7B shows B and M-Mode echocardiogram demonstrating better preservation of contractile function during diastole (green line) and systole (yellow line) in uridine injected animals.

FIG. 7C shows ejection fraction and fractional shortening and left ventricular internal diameter in systole (s) and diastole (d) following uridine administration (mean ±S.E.M. n=15/vehicle and 15/uridine at basal, 1 week, 2 weeks and 3 weeks, n=14/vehicle and 15/uridine at 4 week, **p<0.01).

FIG. 7D shows a pie chart demonstrating fraction of animals with mild, moderate and severe reductions in EF following vehicle or uridine administration.

FIG. 7E shows masson trichrome staining demonstrating scar size (blue) at apex and mid ventricles of vehicle or uridine injected animals.

FIG. 7F shows quantitation of differences in scar size as a fraction of the left ventricular surface area (mean ±S.E.M. n=14/vehicle and 15/uridine, **p<0.01).

FIG. 7G shows a pie chart demonstrating fraction of animals demonstrating mild, moderate and severe fibrosis following vehicle or uridine administration.

FIG. 7H shows heart weight, body weight and HW/BW ratio in vehicle versus uridine treated animals (mean ±S.E.M. n=14/vehicle and 15/uridine, **p<0.01).

FIG. 7I shows histology demonstrating capillaries (CD31 staining) in injured regions of hearts 4 weeks after injury in vehicle or uridine treated animals.

FIG. 7J shows quantitation of capillaries (mean ±S.E.M. n=14/vehicle and 15/uridine, **p<0.01).

FIG. 8A shows experimental design on the use of myricetin in vivo. FIG. 8B shows extracellular ATP hydrolytic activity in injured and uninjured hearts of animals treated with vehicle or myricetin measured after 7 days of myricetin administration post injury demonstrating significant inhibition of ectonucleotidase activity by myricetin (mean ±S.E.M. n=3, **p<0.01).

FIG. 8C shows B and M-mode echocardiogram demonstrating better contractile function in diastole (green line) and systole (yellow line) in hearts of myricetin treated animals.

FIG. 8D shows ejection fraction, fractional shortening and LV chamber size in systole (s) and diastole (d) in vehicle or myricetin treated animals (mean ±S.E.M. n=12/vehicle and 15/myricetin at basal, 1 week and 2 weeks, n=9/vehicle and 14/myricetin at 3 weeks and 4 week, **p<0.01, *p<0.05).

FIG. 8E shows a pie chart illustrating the fraction of animals with mild, moderate and severe reduction in EF at 4 weeks after injury following vehicle or myricetin administration.

FIG. 8F shows masson trichrome staining to demonstrate scar size as a fraction of LV surface area measured 4 weeks after injury at the apex and mid ventricle in vehicle or myricetin injected animals.

FIG. 8G shows quantitation of scar surface area (mean ±S.E.M. n=9/vehicle and 14/myricetin, *p<0.05).

FIG. 8H shows a pie chart illustrating the fraction of animals with mild, moderate and severe fibrosis following vehicle or myricetin administration.

FIG. 8I shows immunostaining demonstrating p53(Ser15 phosphorylation) expression (arrows) in non-myocytes in the injured region of vehicle versus myricetin injected animals and under higher magnification (myocytes are stained by troponin) and quantification (mean S.E.M. n=4, *p<0.05).

FIG. 8J shows gamma H2A.X staining in non-myocyte cells (arrows) in vehicle or myricetin injected animals at 7 days following injury, under higher magnification and quantitation (mean ±S.E.M. n=4, *p<0.05, counts normalized to number of non-myocyte nuclei for I,J).

FIG. 8K shows metabolomic analysis of the hearts of vehicle or myricetin injected animals demonstrating significant increase in pyrimidines uridine, cytidine, decreased carbamoyl aspartate.

FIG. 8L shows decreased adenine+adenosine/uridine ratios in myricetin injected animals compared to vehicle injected animals at 3 days after injury (n=3, *p<0.05, **p<0.01).

FIG. 8M shows metabolomic analysis of serum demonstrating decreased orotate and increased deoxyuridine (day 3) and increased orotidine (day 7) in myricetin injected versus vehicle injected animals (n=3, *p<0.05).

### DETAILED DESCRIPTION

### General

The present disclosure relates to methods and compositions for monitoring ENPP1 activity in a subject (e.g., determining a level of a pyrimidine nucleotide in the subject), treating cardiac injury in a subject (e.g., administering a pyrimidine nucleotide to a subject), and identifying a candidate ENPP1 inhibitor (e.g., contact a cell sample with a test agent and measuring a level of a pyrimidine nucleotide of the cell sample). In certain aspects, the methods and compositions provided herein are based, in part, on the discovery that cardiac cells with increased ENPP1 expression and activity can be effectively treated with a pyrimidine nucleotide, thereby preventing pyrimidine/purine imbalance that eventually leads to cell death. Exemplary pyrimidine nucleotides include uridine, uridine monophosphate (UMP), uridine triphosphate (UTP), cytidine, cytidine monophosphate (CMP), cytidine triphosphate (CTP), orotate, deoxyuridine, and orotidine. Provided herein are methods of measuring levels of certain pyrimidine nucleotides and purine nucleotides (e.g., adenine, adenosine, adenosine monophosphate (AMP), inosine, inosine monophosphate (IMP)) in cardiac cells. In certain aspects, the methods and compositions provided herein may be advantageously used to treat cardiac injury conjointly with another therapeutic agent. For example, in certain embodiments the methods and compositions provided herein may be used to treat cardiac injury conjointly with an ENPP1 inhibitor (e.g., myricetin).

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The term “agent” refers to any substance, compound (e.g., molecule), supramolecular complex, material, or combination or mixture thereof.

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

The term “binding” or “interacting” refers to an association, which may be a stable association, between two molecules, due to, for example, electrostatic, hydrophobic, ionic and/or hydrogen-bond interactions under physiological conditions.

In certain embodiments, therapeutic compounds may be used alone or conjointly administered with another type of therapeutic agent (e.g., additional pyrimidine nucleotide, ENPP1 inhibitor, adenosine kinase inhibitor, adenosine monophosphate deaminase inhibitor). As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of therapeutic compounds with one or more additional therapeutic agent(s) (e.g., one or more additional cardiovascular therapeutic agent(s)) provides improved efficacy relative to each individual administration of the compound (e.g., pyrimidine nucleotide) or the one or more additional therapeutic agent(s). In certain such embodiments, the conjoint administration provides an additive effect, wherein an additive effect refers to the sum of each of the effects of individual administration of the therapeutic compound and the one or more additional therapeutic agent(s).

The term “measuring” refers to determining the presence, absence, quantity amount, or effective amount of a substance in a sample, including the concentration levels of such substances.

As used herein, the term “subject” means a human or non-human animal selected for treatment or therapy.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, the term “cardiomyopathy” refers to any disease or dysfunction of the myocardium (heart muscle) in which the heart is abnormally enlarged, thickened and/or stiffened. As a result, the heart muscle's ability to pump blood is usually weakened. The etiology of the disease or disorder may be, for example, inflammatory, metabolic, toxic, infiltrative, fibroplastic, hematological, genetic, or unknown in origin. There are two general types of cardiomyopathies: ischemic (resulting from a lack of oxygen) and non-ischemic.

As used herein, “chronic heart failure” or “congestive heart failure” or “CHF” refer, interchangeably, to an ongoing or persistent forms of heart failure. Common risk factors for CHF include old age, diabetes, high blood pressure and being overweight. CHF is broadly classified according to the systolic function of the left ventricle as HF with reduced or preserved ejection fraction (HFrEF and HFpEF). The term “heart failure” does not mean that the heart has stopped or is failing completely, but that it is weaker than is normal in a healthy person. In some cases, the condition can be mild, causing symptoms that may only be noticeable when exercising. In others, the condition may be more severe, causing symptoms that may be life-threatening, even while at rest. The most common symptoms of chronic heart failure include shortness of breath, tiredness, swelling of the legs and ankles, chest pain and a cough. In some embodiments, the methods of the disclosure decrease, prevent, or ameliorate one or more symptoms of CHF (e.g., HFrEF) in a subject suffering from or at risk for CHF (e.g., HFrEF). In some embodiments, the disclosure provides methods of treating CHF and conditions that can lead to CHF.

As used herein “acute heart failure” (AHF) or “decompensated heart failure” refer, interchangeably, to a syndrome of the worsening of signs and symptoms reflecting an inability of the heart to pump blood at a rate commensurate to the needs of the body at normal filling pressure. AHF typically develops gradually over the course of days to weeks and then decompensates requiring urgent or emergent therapy due to the severity of these signs or symptoms. AHF may be the result of a primary disturbance in the systolic or diastolic function of the heart or of abnormal venous or arterial vasoconstriction, but generally represents an interaction of multiple factors, including volume overload. The majority of patients with AHF have decompensation of chronic heart failure (CHF) and consequently much of the discussion of the pathophysiology, presentation, and diagnosis of CHF is directly relevant to an understanding of AHF. In other cases, AHF results from an insult to the heart or an event that impairs heart function, such as an acute myocardial infarction, severe hypertension, damage to a heart valve, abnormal heart rhythms, inflammation or infection of the heart, toxins and medications. In some embodiments, the methods of the disclosure decrease, prevent, or ameliorate one or more symptoms of AHF in a subject suffering from or at risk for AHF. In some embodiments, the disclosure provides methods of treating AHF and conditions that can lead to AHF. AHF may be the result of ischemia associated with myocardial infarction.

As used herein the term “cardiac cell” refers to any cell present in the heart that provides a cardiac function, such as heart contraction or blood supply, or otherwise serves to maintain the structure of the heart. Cardiac cells as used herein encompass cells that exist in the epicardium, myocardium or endocardium of the heart. Cardiac cells also include, for example, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure. Cardiac cells may be derived from stem cells, including, for example, embryonic stem cells or induced pluripotent stem cells.

The term “cardiomyocyte” or “cardiomyocytes” as used herein refers to sarcomere-containing striated muscle cells, naturally found in the mammalian heart, as opposed to skeletal muscle cells. Cardiomyocytes are characterized by the expression of specialized molecules e.g., proteins like myosin heavy chain, myosin light chain, cardiac alpha-actinin. The term “cardiomyocyte” as used herein is an umbrella term comprising any cardiomyocyte subpopulation or cardiomyocyte subtype, e.g., atrial, ventricular and pacemaker cardiomyocytes.

As used herein, the phrase “pyrimidine metabolite” refers to a metabolite part of the de-novo synthesis pathway of pyrimidines including carbamoylaspartate, dihydroorotic acid (dihydroorotate), orotic acid, orotidylic acid, orotidine, orotidine monophosphate (01VIP), uridine mono-phosphate (U1VIP), uridine diphosphate (UDP), uridine triphosphate (UTP), TMP, CTP, Uracil, Thymidine, Cytosine.

### Pharmaceutical Compositions and Administration

In certain embodiments, provided herein are pharmaceutical compositions and methods of using pharmaceutical compositions. In some embodiments, the pharmaceutical compositions provided herein comprise a pyrimidine nucleotide (e.g., uridine, uridine monophosphate (UMP), uridine triphosphate (UTP), cytidine, cytidine monophosphate (CMP), cytidine triphosphate (CTP), orotate, deoxyuridine, orotidine). In some embodiments, the pharmaceutical compositions provided herein comprise an ENPP1 inhibitor (e.g., myricetin). In some embodiments, the pharmaceutical compositions provided herein comprise an adenosine kinase inhibitor. In some embodiments, the pharmaceutical compositions provided herein comprise an adenosine monophosphate deaminase inhibitor. In some embodiments, the pharmaceutical compositions provided herein comprise an additional cardiovascular therapeutic agent.

In certain embodiments, the compositions and methods provided herein may be utilized to treat a subject in need thereof. In certain embodiments, the subject is a mammal such as a human, or a non-human mammal. In some embodiments, the subject has myocardial infarction. In certain embodiments, the compositions and methods provided herein may be utilized to promote cardiac wound healing, enhancing cardiac repair, or inhibiting ENPP1 activity a subject in need thereof. In certain embodiments, the compositions and methods provided herein may be utilized to prevent heart failure, cardiac cell death, ectopic calcification of cardiac tissue, scarring of cardiac tissue, or dilated cardiomyopathy in a subject in need thereof. In certain embodiments, the compositions and methods provided herein may be utilized to release of one or more pro-inflammatory molecules from cardiac myocytes in a subject.

When administered to a subject, such as a human, the composition or the compound is preferably administered as a pharmaceutical composition comprising, for example, a therapeutic compound and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In certain embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

In certain embodiments, the pharmaceutical compositions provided herein comprise a pharmaceutically acceptable carrier. The phrase “pharmaceutically acceptable carrier” as used herein means a pharmaceutically acceptable material, composition or vehicle, such as a liquid or solid filler, diluent, excipient, solvent or encapsulating material. A pharmaceutically acceptable carrier can contain physiologically acceptable agents that act, for example, to stabilize, increase solubility or to increase the absorption of a compound. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a therapeutic compound. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In certain embodiments, the pharmaceutical compositions provided herein can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound with the carrier and, optionally, one or more accessory ingredients. In general, the formulations are prepared by uniformly and intimately bringing into association a compound with liquid carriers, or finely divided solid carriers, or both, and then, if necessary, shaping the product.

Formulations suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

In certain embodiments, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular subject, composition, and mode of administration, without being toxic to the subject.

If desired, the effective daily dose of the active compound may be administered as one, two, three, four, five, six or more sub-doses administered separately at appropriate intervals throughout the day, optionally, in unit dosage forms. In certain embodiments, the active compound may be administered two or three times daily. In some embodiments, the active compound will be administered once daily.

In certain embodiments, compounds may be used alone or conjointly administered with another type of therapeutic agent (e.g., an immuno-oncology agent or a chemotherapeutic agent disclosed herein). As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of therapeutic compounds with one or more additional therapeutic agent(s) (e.g., one or more additional chemotherapeutic agent(s)) provides improved efficacy relative to each individual administration of the compound (e.g., copper ionophore) or the one or more additional therapeutic agent(s). In certain such embodiments, the conjoint administration provides an additive effect, wherein an additive effect refers to the sum of each of the effects of individual administration of the therapeutic compound and the one or more additional therapeutic agent(s).

Pharmaceutically acceptable salts of compounds in the methods provided herein. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn, copper, cobalt, cadmium, manganese, or other metal salts.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In some embodiments, the therapeutic compound used in the methods herein is a pyrimidine nucleotide. Exemplary pyrimidine nucleotides are listed in Table 1.

TABLE 1
Exemplary Pyrimidine Nucleotides

| Compound Name | Chemical Structure |
|---|---|
| Uridine | 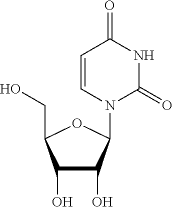 |
| Uridine Monophosphate (UMP) | 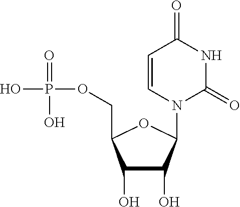 |
| Uridine Triphosphate (UTP) | 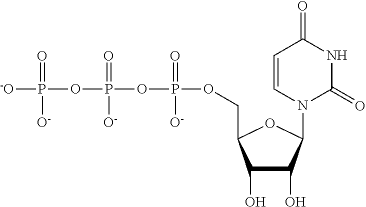 |
| Cytidine | 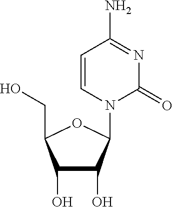 |
| Cytidine monophosphate (CMP) | 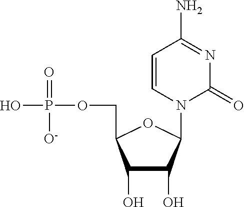 |
| Cytidine Triphosphate (CTP) | 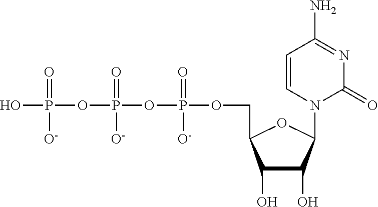 |
| Orotate | 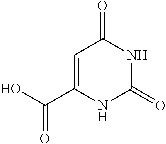 [SMILES: O=C(O)c1cc(=O)[nH]c(=O)[nH]1] |
| Deoxyuridine | 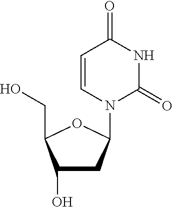 |
| Orotidine | 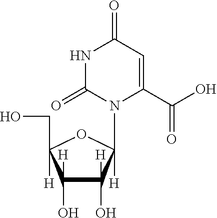 |

In some embodiments, the therapeutic compound is an ENPP1 inhibitor. Exemplary ENPP1 inhibitors are disclosed in U.S. patent application Ser. Nos. 16/193,352, 17/080,093, U.S. Patent Application No. 63/019,773, and U.S. Patent Application No. 63/07,6137, all applications incorporated herein by reference in their entirety, and in particular for their disclosure of ENPP1 inhibitors.

In some embodiments, the ENPP1 inhibitor is a small molecule. In some embodiments, the ENPP1 inhibitor is myricetin.

In some embodiments, the ENPP1 inhibitor is an anti-ENPP1 antibody. In some embodiments, the anti-ENPP1 antibody is a monoclonal antibody. In some embodiments, the anti-ENPP1 antibody is a monoclonal antibody. In some embodiments, the anti-ENPP1 antibody is a polyclonal antibody. Exemplary anti-ENPP1 antibodies are further disclosed in U.S. Patent Application No. 63/01,9773, incorporated herein by reference in its entirety, and in particular for its disclosure of anti-ENPP1 antibodies.

In some embodiments, the therapeutic compound is an adenosine kinase inhibitor.

In some embodiments, the therapeutic compound is an adenosine monophosphate deaminase inhibitor.

In some embodiments, the therapeutic compound is an additional cardiovascular therapeutic agent. Exemplary classes of additional cardiovascular therapeutic agents include beta blockers, ACE inhibitors, angiotensin receptor blockers, aldosterone antagonist, digoxin, hydralazine and nitrates, and diuretics. Examples of additional cardiovascular therapeutic agents include, but are not limited to, sulfaphenazole, chloramphenicol, statins, metformin, resveratrol, minoxidil, clonidine, amiodarone, intermedin, enalapril, candesartan, spironolactone, pravastin, atorvastin, dexrazoxane, aspirin, enoxaparin, rivaroxaban/apixaban, carvedilol, nebivolol, metoprolol, bisoprilol, lisinopril, captopril, losartan, entresto, sacubitril/valsartan, spironolactone, eplerenone, Apresoline, Nitrobid, Imdur, Isordil, furosemide (Lasix), bumetanide (Bumex), torsemide (Demadex), and metolazone (Zaroxolyn).

### Methods of Monitoring ENPP1 Activity

ENPP1 (Ectonucleotide pyrophosphatase/phosphodiesterase 1) is a type II transmembrane protein, that hydrolyzes extracellular ATP into AMP and PPi (pyrophosphate). ENPP1 plays a role in ectopic calcification, an extreme form of dysregulated wound healing. ENPP1 expression is dramatically upregulated in an infarcted heart and is predominantly expressed by cardiac fibroblasts. ATP is a damage associated molecular pattern (DAMP) signal associated with acute injury. Extracellular ATP concentration in the uninjured heart is low but rises by orders of magnitude after cardiac injury. Deletion of ENPP1 by genetic means leads to a profound improvement in post infarct wound healing with decreases inflammation, and results in significantly better cardiac function. Furthermore, inhibition of ENPP1 with an ENPP1 inhibitor augments wound healing in the heart after myocardial infarction, reducing inflammation and leads to better preservation of post injury heart function.

ENPP1 mediates cleavage of ATP into AMP and PPi signals to myocytes, which release small molecules/metabolites that are pro-inflammatory and induce cell death of a variety of non-myocyte cells including fibroblasts, macrophages, endothelial cells and smooth muscle cells. AMP that is formed by ENPP1 mediated hydrolysis of ATP induces cardiomyocytes to release purine nucleotides that induce cell death of non-myocytes. Release of purine nucleotides disrupts pyrimidine biosynthesis of proliferating non-myocyte cells, induces genotoxic stress and initiates a p53 mediated DNA damage response that results in cell cycle arrest and apoptosis. Macrophages, endothelial cells and fibroblasts are critical components of the cardiac wound healing process and depletion or functional impairment of non-myocyte cells is known to worsen cardiac wound healing. A nucleotide balance between the content of purines and pyrimidines available to cycling cells is critical to avoid genotoxic stress and maintain genomic stability. Defects in pyrimidine biosynthesis result in insufficient pyrimidine precursors lead to a DNA damage response in cycling non-myocytes and resulting in cell death. The imbalance of purines/pyrimidines is a key event initiating the cell cycle arrest/apoptotic cascade as supplementation of uridine to correct decreased pyrimidine levels rescues cell death.

Administered of pyrimidine nucleotides to animals after ischemic cardiac injury rescues pyrimidine biosynthesis in non-myocyte cells and results significant improvement in wound healing and post injury cardiac function.

Purines (e.g., adenine) and pyrimidines (e.g., cytosine) are two classes of nucleotides which forms nucleic acids (e.g., DNA and RNA). Apart from the primary role of DNA and RNA as “genetic information storage”, nucleotides also serves different functions in the cells such as energy carrier (e.g., ATP and GTP), components of co-enzymes (e.g., NAD and FAD), and cellular signal transduction (cAMP and cGMP as ‘second messengers’). An ample supply of nucleotides in the cell is very essential for all cellular processes. Pyrimidines have diverse biological activities such as antimicrobial, CNS depressant, anti-inflammatory, analgesic, anti-convulsant, anticancer, antihelmentic, antioxidant and herbicidal.

Pyrimidine is synthesized as a free ring and then a ribose-5-phosphate is added to yield direct nucleotides, whereas, in purine synthesis, the ring is made by attaching atoms on ribose-5-phosphate. Biosynthesis of pyrimidine nucleotides takes places in the cytoplasm and can occur by a de novo pathway or by the reutilization of preformed pyrimidine bases or ribonucleosides (salvage pathway).

In the de novo synthesis of pyrimidines, the ring is synthesized first and then it is attached to a ribose-phosphate to for a pyrimidine nucleotide. CO₂ and glutamine are combined to form carbamoyl phosphate. This reaction is catalyzed by carbamoyl phosphate synthetase II, which is the major regulated step for this pathway. Carbamoyl phosphate is then combined with water and aspartate before being subsequently dehydrogenated in a series of reactions to form orotic acid. The ribose-5-phosphate ring is then attached to orotic acid by orotate phosphoribosyl transferase to form Orotidine 5′-monophosphate (OMP). OMP is decarboxylated to form UMP by OMP decarboxylase. UMP can then be phosphorylated to form UTP. UTP can subsequently be converted to CTP with the addition of an amino group that is donated by glutamine. The conversion of UTP to CTP is catalyzed by CTP synthetase.

Pyrimidines can be salvaged from orotic acid, uracil, and thymine but not from cytosine. Salvage is accomplished by the enzyme pyrimidine phosphoribosyl transferase. Deficiencies in orotate phosphoribosyl transferase or OMP decarboxylase can lead to orotic aciduria which is characterized by growth retardation and anemia.

Biosynthesis of purine nucleotides can occur by two pathways: de novo synthesis pathway, and salvage pathway. The de novo synthesis of purine nucleotide involves using phosphoribose, amino acid, one carbon units and CO2 as raw materials to synthesize purine nucleotide from the beginning. First, Ribose-5-phosphate (as provided by the pentose-phosphate pathway) is converted into PRPP (Phosphoribosyl pyrophosphate) by PRPP synthetase, in a step requiring one ATP. Next, an α-amino group is then added to PRPP from glutamine to form 5-phosphoribosylamine. This reaction is catalyzed by glutamine PRPP amidinotransferase.

A series of nine reactions results in the formation of IMP (Inosine 5′-monophosphate). IMP can then be transformed either to GMP by IMP dehydrogenase, or to AMP by adenylosuccinate synthetase.

Bases from degraded nucleic acids can be converted back into purine nucleotides via the salvage pathways. Hypoxanthine can be combined with PRPP (which acts as the donor of ribose-5 phosphate) to form IMP in a reaction catalyzed by Hypoxanthine-guanine phosphoribosyltransferase (HGPRT). IMP can subsequently be transformed into AMP or GMP via the last few steps of the pathway of de novo purine synthesis. HGPRT also catalyzes the reaction which combines PRPP with guanine to form GMP. Adenine phosphoribosyltransferase converts adenine and PRPP to form AMP.

In certain aspects, provided herein are methods related to monitoring ENPP1 activity that comprise determining whether a level of a pyrimidine nucleotide in serum of the subject is below a threshold level, wherein a level of the pyrimidine nucleotide below the threshold level is indicative of ENPP1 activity. In certain embodiments, the pyrimidine nucleotide is involved in pyrimidine biosynthesis. In certain embodiments, the pyrimidine nucleotide prevents cardiac cell death. In certain embodiments, determining whether the level of the pyrimidine nucleotide is below a threshold level comprises measuring the level of the pyrimidine nucleotide in the serum. Exemplary pyrimidine nucleotides are listed in Table 1.

In certain aspects, provided herein are methods related to monitoring ENPP1 activity after cardiac injury in a subject comprising (a) determining a level of a purine nucleotide and/or a purine nucleoside in serum of the subject; (b) determining a level of a pyrimidine nucleotide in serum of the subject; and (c) determining whether a ratio of the level of the purine nucleotide and/or the purine nucleoside to the level of the pyrimidine nucleotide is above a threshold level, wherein a ratio above the threshold level is indicative of ENPP1 activity. In certain embodiments, the pyrimidine nucleotide is involved in pyrimidine biosynthesis. In certain embodiments, the pyrimidine nucleotide prevents cardiac cell death. In certain embodiments, determining whether the level of the pyrimidine nucleotide is below a threshold level comprises measuring the level of the pyrimidine nucleotide in the serum. In certain embodiments, the purine nucleotide and/or the purine nucleoside disrupts pyrimidine biosynthesis. In certain embodiments, the purine nucleotide and/or the purine nucleoside induces cardiac cell death. In certain embodiments, determining a level of a purine nucleotide and/or a purine nucleoside in serum of the subject comprises measuring the level of the purine nucleotide and/or purine nucleoside of the serum. Exemplary purine nucleotides and nucleosides are listed in Table 2. Inhibiting ENPP1 thus rescues pyrimidine biosynthesis at the orotidine biosynthesis step. Accordingly, orotidine, orotidine mono phosphate, uridine and cytidine or its mono, di and tri phosphates become circulating biomarkers that can be measured to assess the effectiveness of ENPP1 inhibition. In particular, orotidine and orotate levels will increase following ENPP1 inhibition, with the degree of elevation proportional to the degree of inhibition.

TABLE 2
Exemplary Purine Nucleotides and Nucleosides

| Gene Name | Structure |
|---|---|
| Adenine | 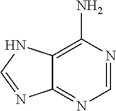 |
| Adenosine | 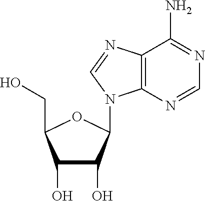 |
| Adenosine monophosphate (AMP) | 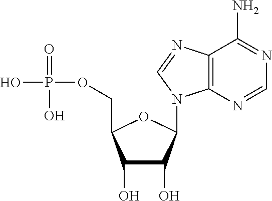 |
| Inosine | 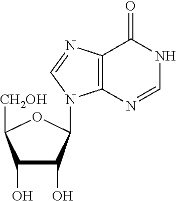 |
| Inosine monophosphate (IMP) | 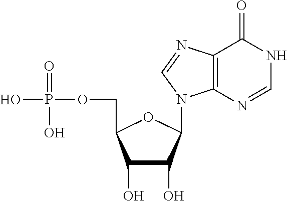 |

In certain aspects, provided herein are methods related to treating myocardial infarction, promoting cardiac wound healing, enhancing cardiac repair, inhibiting ENPP1 activity, and/or preventing heart failure, cardiac cell death, ectopic calcification of cardiac tissue, scarring of cardiac tissue, dilated cardiomyopathy, and/or release of one or more pro-inflammatory molecules from cardiac myocytes in a subject, comprising: (a) determining whether serum of the subject comprises a level of a pyrimidine nucleotide below a threshold level; and (b) if the serum is characterized by a level below the threshold level, administering the pyrimidine nucleotide to the subject. In certain embodiments, the pyrimidine nucleotide is involved in pyrimidine biosynthesis. In certain embodiments, the pyrimidine nucleotide prevents cardiac cell death. In certain embodiments, determining whether the level of the pyrimidine nucleotide is below a threshold level comprises measuring the level of the pyrimidine nucleotide in the serum.

In certain aspects, provided herein are methods related to treating myocardial infarction, promoting cardiac wound healing, enhancing cardiac repair, inhibiting ENPP1 activity, and/or preventing heart failure, cardiac cell death, ectopic calcification of cardiac tissue, scarring of cardiac tissue, dilated cardiomyopathy, and/or release of one or more pro-inflammatory molecules from cardiac myocytes in a subject, comprising: (a) determining a level of a purine nucleotide and/or a purine nucleoside in serum of the subject; (b) determining a level of a pyrimidine nucleotide in serum of the subject; (c) determining whether a ratio of the level of the purine nucleotide and/or the purine nucleoside to the level of the pyrimidine nucleotide is above a threshold level; and (d) if the ratio of the level of the purine nucleotide and/or the purine nucleoside to the level of the pyrimidine nucleotide is above a threshold level, administering the pyrimidine nucleotide to the subject. In certain embodiments, the pyrimidine nucleotide is involved in pyrimidine biosynthesis. In certain embodiments, the pyrimidine nucleotide prevents cardiac cell death. In certain embodiments, determining whether the level of the pyrimidine nucleotide is below a threshold level comprises measuring the level of the pyrimidine nucleotide in the serum. In certain embodiments, the purine nucleotide and/or the purine nucleoside disrupts pyrimidine biosynthesis. In certain embodiments, the purine nucleotide and/or the purine nucleoside induces cardiac cell death. In certain embodiments, determining a level of a purine nucleotide and/or a purine nucleoside in serum of the subject comprises measuring the level of the purine nucleotide and/or purine nucleoside of the serum.

In certain embodiments, the threshold level of uridine in serum of a subject is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the serum comprise uridine.

In certain embodiments, the threshold level of uridine monophosphate (UMP) in serum of a subject is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the serum comprise uridine monophosphate (UMP).

In certain embodiments, the threshold level of uridine triphosphate (UTP) in serum of a subject is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the serum comprise uridine triphosphate (UTP).

In certain embodiments, the threshold level of cytidine in serum of a subject is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the serum comprise cytidine.

In certain embodiments, the threshold level of cytidine monophosphate (CMP) in serum of a subject is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the serum comprise cytidine monophosphate (CMP).

In certain embodiments, the threshold level of cytidine triphosphate (CTP) in serum of a subject is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the serum comprise cytidine triphosphate (CTP).

In certain embodiments, the threshold level of orotate in serum of a subject is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the serum comprise orotate.

In certain embodiments, the threshold level of deoxyuridine in serum of a subject is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the serum comprise deoxyuridine.

In certain embodiments, the threshold level of orotidine in serum of a subject is met if at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100% of the serum comprise orotidine.

In certain embodiments, the threshold ratio of a level of a purine to a level uridine nucleotide in serum of a subject is met if the ratio is at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100%.

In certain embodiments, the threshold ratio of a level of a purine to a level uridine monophosphate (UMP) nucleotide in serum of a subject is met if the ratio is at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100%.

In certain embodiments, the threshold ratio of a level of a purine to a level uridine triphosphate (UTP) in serum of a subject is met if the ratio is at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100%.

In certain embodiments, the threshold ratio of a level of a purine to a level cytidine nucleotide in serum of a subject is met if the ratio is at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100%.

In certain embodiments, the threshold ratio of a level of a purine to a level cytidine monophosphate (CMP) nucleotide in serum of a subject is met if the ratio is at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100%.

In certain embodiments, the threshold ratio of a level of a purine to a level is cytidine triphosphate (CTP) nucleotide in serum of a subject is met if the ratio is at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100%.

In certain embodiments, the threshold ratio of a level of a purine to a level orotate nucleotide in serum of a subject is met if the ratio is at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100%.

In certain embodiments, the threshold ratio of a level of a purine to a level deoxyuridine nucleotide in serum of a subject is met if the ratio is at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100%.

In certain embodiments, the threshold ratio of a level of a purine to a level orotidine nucleotide in serum of a subject is met if the ratio is at least 0.5%, 0.6%, 0.7%, 0.8%, 0.9%, 1.0%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 81%, 82%. 83%, 84%, 85%, 86%, 87%, 88%, 89%, 90%, 91%, 92%, 93%, 94%, 95%, 96%, 97%, 98%, 99% or 100%.

In some embodiments, any assay capable of detecting levels of the relevant pyrimidine nucleotide and/or purine nucleotide (a biomarker) can be used in the methods provided herein. In some embodiments, the pyrimidine nucleotide and/or purine nucleotide is detected by immunostaining with a labeled antibody that binds to the biomarker epitope. In some embodiments, the biomarker is detected by immunohistochemistry. In some embodiments, the biomarker is detected by Western Blot. In some embodiments, the mRNAs of the biomarker are detected using qPCR. In some embodiments, the biomarker is detected using fluorescence activated cell sorting (FACS). In some embodiments, the biomarker is detected using microscopy (e.g., fluorescence microscopy). In some embodiments, the biomarker is detected using ELISA.

Any of a variety of antibodies can be used in methods of the detection. Such antibodies include, for example, polyclonal, monoclonal (mAbs), recombinant, humanized or partially humanized, single chain, Fab, and fragments thereof. The antibodies can be of any isotype, e.g., IgM, various IgG isotypes such as IgG1, IgG2a, etc., and they can be from any animal species that produces antibodies, including goat, rabbit, mouse, chicken or the like. The term “an antibody specific for” a protein means that the antibody recognizes a defined sequence of amino acids, or epitope, in the protein, and binds selectively to the protein and not generally to proteins unintended for binding to the antibody. The parameters required to achieve specific binding can be determined routinely, using conventional methods in the art.

In some embodiments, antibodies specific for a biomarker (e.g., pyrimidine nucleotide and/or purine nucleotide) are immobilized on a surface (e.g., are reactive elements on an array, such as a microarray, or are on another surface, such as used for surface plasmon resonance (SPR)-based technology, such as Biacore), and proteins in a sample are detected by virtue of their ability to bind specifically to the antibodies. Alternatively, proteins in the sample can be immobilized on a surface, and detected by virtue of their ability to bind specifically to the antibodies. Methods of preparing the surfaces and performing the analyses, including conditions effective for specific binding, are conventional and well-known in the art.

Among the many types of suitable immunoassays are immunohistochemical staining, ELISA, Western blot (immunoblot), immunoprecipitation, radioimmunoassay (MA), fluorescence-activated cell sorting (FACS), etc. In some embodiments, assays used in methods provided herein can be based on colorimetric readouts, fluorescent readouts, mass spectroscopy, visual inspection, etc.

As mentioned above, expression levels of a biomarker can be measured by measuring nucleic acid amounts (e.g., mRNA amounts and/or genomic DNA). The determination of nucleic acid amounts can be performed by a variety of techniques known to the skilled practitioner. For example, expression levels of nucleic acids, alternative splicing variants, chromosome rearrangement and gene copy numbers can be determined by microarray analysis (see, e.g., U.S. Pat. Nos. 6,913,879, 7,364,848, 7,378,245, 6,893,837 and 6,004,755) and quantitative PCR. Copy number changes may be detected, for example, with the Illumina Infinium II whole genome genotyping assay or Agilent Human Genome CGH Microarray (Steemers et al., 2006). Examples of methods to measure mRNA amounts include reverse transcriptase-polymerase chain reaction (RT-PCR), including real time PCR, microarray analysis, nanostring, Northern blot analysis, differential hybridization, and ribonuclease protection assay. Such methods are well-known in the art and are described in, for example, Sambrook et al., Molecular Cloning: A Laboratory Manual, current edition, Cold Spring Harbor Laboratory, Cold Spring Harbor, N.Y., and Ausubel et al., Current Protocols in Molecular Biology, John Wiley & sons, New York, N.Y.

### Methods of Treating or Preventing Cardiac Injury

Heart failure (HF) is a complex clinical syndrome that can result from any structural or functional cardiovascular disorder causing systemic perfusion inadequate to meet the body's metabolic demands without excessively increasing left ventricular filling pressures. It is characterized by specific symptoms, such as dyspnea and fatigue, and signs, such as fluid retention. Calcification of soft tissues is a cell mediated process that resembles bone formation in the skeletal system with calcification of the extracellular matrix by cells capable of mineralization. Analogous to bone formation, osteogenic cells are thought to be recruited to the affected tissue and induce mineralization. Pathological mineralization of soft tissues, or ectopic calcification, commonly occurs with tissue injury and degeneration and in common diseases such as diabetes and chronic kidney disease.

In the heart, calcification of cardiac muscle leads to conduction system disturbances and is one of the most common pathologies underlying heart blocks. Calcification of the cardiovascular system is associated with more than 100-500 fold increase in cardiovascular mortality. Myocardial calcification is observed in the aging heart and in patients with diabetes, renal disease, and myocardial injury secondary to ischemia or inflammation. Cardiac pump dysfunction and arrhythmias can also occur depending on the extent and anatomic site of calcification and calcified myocardial scars have been reported to cause refractory ventricular tachycardia. Cardiac calcification is also a prognostic indicator of poor outcomes following myocardial infarction or myocarditis.

In certain aspects, provided herein are methods of treating or preventing cardiac injury in a subject by administering to the subject a therapeutic compound according to a method provided herein. In certain embodiments, the therapeutic compound is a pyrimidine nucleotide.

In certain embodiments, the compositions and methods provided herein may be utilized to treat myocardial infarction, promote cardiac wound healing, enhance cardiac repair, inhibit ENPP1 activity, and/or prevent heart failure, cardiac cell death, ectopic calcification of cardiac tissue, scarring of cardiac tissue, dilated cardiomyopathy, and/or release of one or more pro-inflammatory molecules from cardiac myocytes.

In certain embodiments, the compositions and methods provided herein may be utilized to promote cardiac wound healing, enhancing cardiac repair, or inhibiting ENPP1 activity a subject in need thereof In certain embodiments, the compositions and methods provided herein may be utilized to prevent heart failure, cardiac cell death, ectopic calcification of cardiac tissue, scarring of cardiac tissue, or dilated cardiomyopathy in a subject in need thereof. In certain embodiments, the compositions and methods provided herein may be utilized to release of one or more pro-inflammatory molecules from cardiac myocytes in a subject.

Actual dosage levels of the therapeutic compound may be varied so as to obtain an amount which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

### Methods of Screening Candidate ENPP1 Inhibitors

Certain aspects of the disclosure are directed to a method of screening one or more test agents to identify a candidate ENPP1 inhibitor, comprising contacting a cell sample (e.g., cardiac cell) with a test agent, measuring a level of a pyrimidine nucleotide of the cell sample (e.g., uridine, UMP, UTP, cytidine, CMP, CTP, orotate, deoxyuridine, orotidine) and identifying the test agent as a candidate ENPP1 inhibitor if the level of the pyrimidine nucleotide is increased as compared to a level of pyrimidine nucleotide of a corresponding cell sample not contacted with the test agent. The level of a pyrimidine nucleotide of a corresponding cell sample not contacted with the test agent can be any suitable reference, such as a control sample or a reference sample (which in some embodiments may be representative of normal pyrimidine biosynthesis, and in other embodiments may be representative of increased pyrimidine biosynthesis.

In some embodiments of the invention, the test agent is identified as a candidate ENPP1 inhibitor if a level of the a pyrimidine nucleotide (e.g., uridine, UMP, UTP, cytidine, CMP, CTP, orotate, deoxyuridine, orotidine) is increased by at least about 5%, 10%, 20%, 30%, 40%, 50%, 60%, 75%, 90%, 99% or more. In some embodiments of the invention, the test agent is identified as a candidate ENPP1 inhibitor if a level of the pyrimidine nucleotide (e.g., uridine, UMP, UTP, cytidine, CMP, CTP, orotate, deoxyuridine, orotidine) is increased by at least 1-fold, 2-fold, 3-fold, 5-fold, 6-fold, 7-fold, 8-fold, 9-fold, 10-fold or more.

In some embodiments, the method further comprises measuring cell death of the contacted cell sample and determining if cell death of the contacted cell is decreased as compared to cell death of a corresponding cell sample not contacted with the test agent.

In some embodiments, any assay capable of detecting cell death after treatment with a test agent can be used in the methods provided herein. Cell death is typically characterized by membrane blebbing, condensation of cytoplasm, and the activation of endogenous endonucleases.

Other aspects of the disclosure are directed to a method of screening one or more test agents to identify a candidate ENPP1 inhibitor, comprising (a) contacting a cell sample with a test agent; (b) measuring a level of a purine nucleotide and/or the purine nucleoside of the sample; (c) measuring a level of a pyrimidine nucleotide of the cell sample; (d) determining a ratio of the level of the purine nucleotide and/or the purine nucleoside to the level of the pyrimidine nucleotide; and (e) identifying the test agent as a candidate ENPP1 inhibitor if the ratio is decreased as compared to a ratio of the level of the purine nucleotide and/or the purine nucleoside to the level of the pyrimidine nucleotide of a cell sample not contacted with the test agent. The ratio of the level of the purine nucleotide and/or the purine nucleoside to the level of the pyrimidine nucleotide of a corresponding cell sample not contacted with the test agent can be any suitable reference, such as a control sample or a reference sample (which in some embodiments may be representative of normal pyrimidine biosynthesis, and in other embodiments may be representative of increased pyrimidine biosynthesis.

In some embodiments of the invention, the test agent is identified as a candidate ENPP1 inhibitor if the ratio of the level of the purine nucleotide and/or the purine nucleoside to the level of the pyrimidine nucleotide is decreased by at least about 5%, 10%, 20%, 30%, 40%, 50%, 60%, 75%, 90%, 99% or more. In some embodiments of the invention, the test agent is identified as a candidate ENPP1 inhibitor if the ratio of the level of the purine nucleotide and/or the purine nucleoside to the level of the pyrimidine nucleotide is decreased by at least 1-fold, 2-fold, 3-fold, 5-fold, 6-fold, 7-fold, 8-fold, 9-fold, 10-fold or more.

In some embodiments, the method further comprises measuring cell death of the contacted cell sample and determining if cell death of the contacted cell is decreased as compared to cell death of a corresponding cell sample not contacted with the test agent.

In some embodiments, any assay capable of detecting cell death after treatment with a test agent can be used in the methods provided herein. Cell death is typically characterized by membrane blebbing, condensation of cytoplasm, and the activation of endogenous endonucleases.

Cell viability can be measured by determining in a cell the uptake of a dye such as neutral red, trypan blue, or ALAMAR™ blue (see, e.g., Page et al., 1993, Intl. J. Oncology 3:473-476). In such an assay, the cells are incubated in media containing the dye, the cells are washed, and the remaining dye, reflecting cellular uptake of the dye, is measured spectrophotometrically. The protein-binding dye sulforhodamine B (SRB) can also be used to measure cytoxicity (Skehan et al., 1990, J. Natl. Cancer Inst. 82:1107-12).

Alternatively, a tetrazolium salt, such as MTT, is used in a quantitative colorimetric assay for mammalian cell survival and proliferation by detecting living, but not dead, cells (see, e.g., Mosmann, 1983, J. Immunol. Methods 65:55-63).

Cell death can be quantitated by measuring, for example, DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, 1999, no. 2, pp. 34-37 (Roche Molecular Biochemicals).

Cell death can also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring cell death number has been described by Duke and Cohen, Current Protocols in Immunology (Coligan et al. eds., 1992, pp. 3.17.1-3.17.16). Cells also can be labeled with a DNA dye (e.g., acridine orange, ethidium bromide, or propidium iodide) and the cells observed for chromatin condensation and margination along the inner nuclear membrane. Other morphological changes that can be measured to determine cell death include, e.g., cytoplasmic condensation, increased membrane blebbing, and cellular shrinkage.

The presence of cell death can be measured in both the attached and “floating” compartments of the cultures. For example, both compartments can be collected by removing the supernatant, trypsinizing the attached cells, combining the preparations following a centrifugation wash step (e.g., 10 minutes at 2000 rpm), and detecting cell death (e.g., by measuring DNA fragmentation). (See, e.g., Piazza et al., 1995, Cancer Research 55:3110-16).

### EXEMPLIFICATION

Organ metabolism is spatio-temporally regulated at the cellular and tissue level to link metabolic pathways with key homeostatic processes, but little is known about the cellular regulation of metabolism during tissue repair after acute injury. In a murine model of ischemic cardiac injury, it was demonstrated that cardiac muscle cell regulates pyrimidine biosynthesis of non-muscle cells to affect cardiac repair. It was demonstrated that the ectonucleotidase ENPP1 hydrolyzes extracellular ATP released after cardiac injury to form AMP, which then induces the cardiomyocyte to release adenine and specific ribonucleosides that disrupt pyrimidine biosynthesis, cause genotoxic stress and induce a p53 mediated cell death of non-myocyte cells such as fibroblasts, macrophages, endothelial and smooth muscle cells. As non-myocyte cells play a critical role in mediating heart repair, it was demonstrated that rescue of pyrimidine biosynthesis by administration of uridine after cardiac injury or by genetic targeting of ENPP1/AMP pathway enhances repair and post infarct heart function. A high through-put assay was established to screen a large library of small molecules to identify small molecule ENPP1 inhibitors and demonstrate that systemic administration of ENPP1 inhibitors following heart injury rescues pyrimidine biosynthesis in non-myocyte cells and augments tissue repair and function. Specific biochemical steps of pyrimidine biosynthesis that are disrupted were determined and critical pyrimidine metabolite orotidine was identified as a serum biomarker for monitoring the metabolic control of tissue repair. These observations demonstrate that the cardiac muscle cell by releasing adenine regulates pyrimidine metabolism in non-muscle cells via paracrine mechanisms and provide insight into how inter-cellular regulation of pyrimidine biosynthesis can be targeted and monitored for augmenting tissue repair.

Metabolism of organs is tightly regulated in a spatio-temporal manner both at the cellular and tissue level to link metabolic pathways with key biological processes such as cellular proliferation, differentiation and signaling[1]. The metabolic state of the cell is known to function as a checkpoint during cell division and spatial compartmentalization of metabolic pathways provides efficient maintenance of tissue homeostasis [2]. For instance, differences in glycolytic activity between neurons and astrocytes establish an astrocyte-neuron lactate shuttle that enables neurons to maintain cellular redox balance and viability [3]. However, little is understood about how metabolic pathways regulate tissue repair. After acute heart injury, different cell types are recruited to the injured tissue in a spatio-temporally regulated manner to contribute to wound healing. An initial polymorphonuclear infiltrate in replaced by macrophages and vital cues provided by inflammatory cells initiate fibroblast and endothelial cell proliferation to form granulation tissue [4, 5] that subsequently matures to form a healthy scar [6]. The necrotic and inflamed tissues represent a stressful environment for both parenchymal cells and non-parenchymal cells and how cellular metabolism is spatially regulated and affects tissue repair after acute injury remains an unanswered question.

It was demonstrated that cardiac muscle cells disrupt pyrimidine biosynthesis of non-muscle cells in the injured region thereby impacting tissue repair. It was shown that such a disruption of pyrimidine biosynthesis in non-myocyte cells by paracrine mechanisms interferes with wound healing but can be pharmacologically targeted to augment tissue repair and organ function. Following ischemic cardiac injury, the extracellular ATP levels increase in the injured region from extravasation of intracellular ATP from necrotic myocytes, increased membrane permeability and upregulated activity of nucleotide transporters[7-9]. ATP is a damage associated molecular pattern (DAMP) signal associated with acute injury[10]. Ectonucleotidases are membrane bound enzymes with an extracellular catalytic domain that hydrolyzes extracellular ATP. It was shown that the ectonucleotidase ENPP1 [11] is induced in non-myocyte cells after cardiac injury and is the principal nucleotidase that hydrolyzes extracellular ATP in the injured heart. Using in vitro and in vivo approaches, it was shown that AMP that is formed by ENPP1 mediated hydrolysis of ATP induces the cardiomyocytes to release adenine base and specific ribonucleosides that in combination induce cell death of non-myocytes. It was shown that the combination of adenine and specific ribonucleosides released by cardiomyocytes, disrupts pyrimidine biosynthesis of proliferating non-myocyte cells, induces genotoxic stress and initiates a p53 mediated DNA damage response that results in cell cycle arrest and apoptosis. Macrophages, endothelial cells and fibroblasts are critical components of the cardiac wound healing process and depletion or functional impairment of non-myocyte cells is known to worsen cardiac wound healing[12]. As cardiomyocyte secreted metabolites disrupt pyrimidine biosynthesis in non-myocytes, the pyrimidine uridine was systemically administered to animals after ischemic cardiac injury to rescue pyrimidine biosynthesis in non-myocyte cells and demonstrate significant improvement in wound healing and post injury cardiac function. As the ENPP1/AMP pathway initiates this cascade of events, it was shown with genetic loss of function approaches that conditional deletion of ENPP1 at the time of ischemic cardiac injury is associated with a significant improvement in post MI wound healing and heart function. A systems genetics approach across 100 inbred strains of mice subjected to cardiac injury also demonstrated the importance of the ENPP1/AMP pathway in determining post injury outcomes. A large library of small molecules was screened to identify ENPP1 inhibitors and demonstrate that systemic administration of an ENPP1 inhibitor following ischemic cardiac injury rescues pyrimidine biosynthesis in non-myocytes, attenuates a p53 mediated DNA damage response and leads to augmented wound healing. Specific pyrimidine biosynthetic steps that are disrupted were identified and it was demonstrated that serum levels of orotidine, a key pyrimidine metabolite whose synthesis is disrupted by the ENPP1/AMP mediated cascade can serve as a blood biomarker to monitor pyrimidine biosynthesis and its successful targeting during tissue repair. These observations demonstrate an inter-cellular regulation of pyrimidine biosynthesis after tissue injury and how such defects in pyrimidine biosynthesis can be specifically targeted to enhance cardiac repair and post injury heart function.

### Example 1: ENPP1 Expression Increases by an Order of Magnitude Early after Cardiac Injury and is the Principal Nucleotidase that Hydrolyzes Extracellular ATP

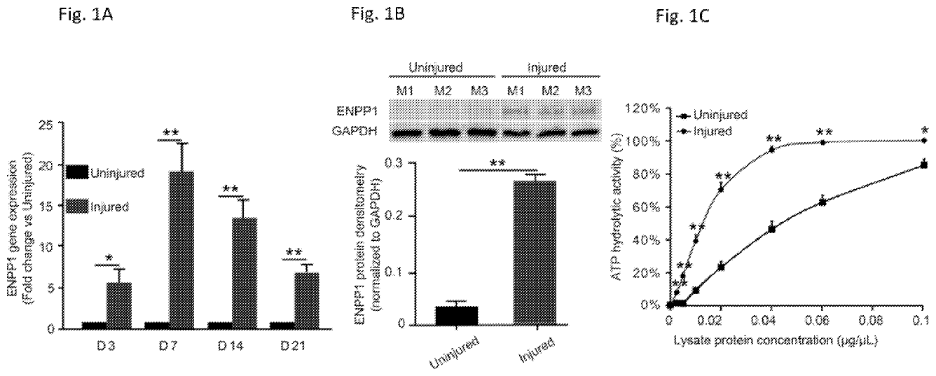

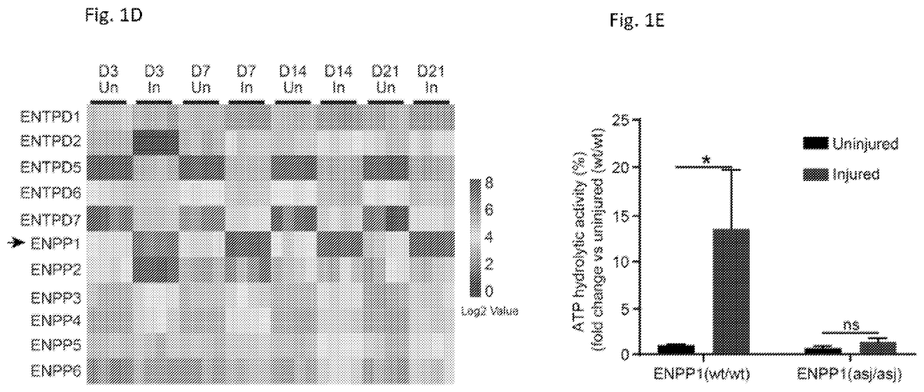

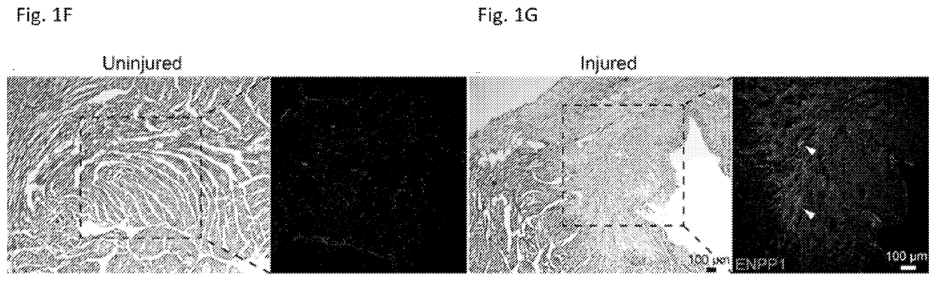

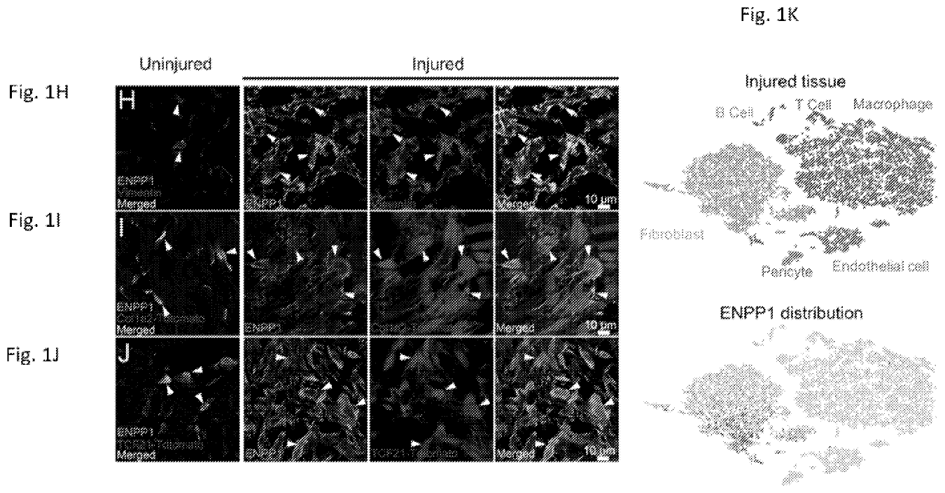

Both male and female adult mice (C57BL/6J) were subjected to ischemic cardiac injury by permanent ligation of the left anterior descending coronary artery that supplies the bulk of blood flow to the left ventricle. The hearts were harvested at 3,7,14 and 21 days after ischemic injury, dissected the injured and uninjured regions of the same heart and performed qPCR to determine temporal changes in ENPP1 gene expression. ENPP1 expression increased 5-fold on Day 3 after injury and was approximately 15-20 fold higher by Day 7 as compared to uninjured regions (FIG. 1A).

Thereafter, ENPP1 expression declined albeit still remaining elevated at 21 days after cardiac injury (FIG. 1A). Western Blotting confirmed increased ENPP1 protein expression in the injured regions of hearts at 7 days following injury (FIG. 1B). To determine whether increased ENPP1 protein in the injured heart was associated with increased ENPP1 enzymatic activity, an enzymatic activity assay was performed where uninjured and injured regions of the heart were homogenized and the ability of the tissue homogenate to hydrolyze extracellular ATP was measured by a luminescence assay. It was observed that at similar concentrations of tissue lysate, ATP hydrolytic activity was significantly increased in the injured heart regions, suggesting that increased ENPP1 protein was associated with increased ectonucleotidase activity (FIG. 1C). As there are several members of the ENPP1 and ectonucleotidase family[13, 14] that hydrolyze extracellular ATP, RNA-seq data sets of acute cardiac injury were analyzed[15] and it was observed that of the known mammalian ectonucleotidases that hydrolyze ATP, ENPP1 was the only one that demonstrated the most early, robust and consistent increase in expression after cardiac injury (FIG. 1D). To confirm that increased ATP hydrolytic activity in injured cardiac tissue samples is predominantly due to increased ENPP1 enzymatic activity, ENPP1 mutant mice (ENPP1asj/asj mice) were subjected to ischemic cardiac injury. The ENPP1asj/asj mice have an amino acid substitution in the catalytic domain that renders the catalytic domain devoid of ATP hydrolytic activity[16]. Injured cardiac tissue harvested from wild type mice demonstrated increased ATP hydrolytic activity but injured heart tissue harvested from ENPP1asj/asj animals did not show any increase in the ability to hydrolyze ATP (FIG. 1E). Taken together these observations demonstrate that ENPP1 expression and activity increase significantly in the injured heart and that ENPP1 is the principal nucleotidase that hydrolyzes extracellular ATP after cardiac injury.

Next, the spatial expression of ENPP1 in the injured heart was examined by immunostaining and it was observed that ENPP1 was minimally expressed in the uninjured region (FIG. 1F), but following injury, there was a robust increase in ENPP1 expression predominantly restricted to the injury region (FIG. 1G). To identify the phenotype of the cell expressing ENPP1, the injured segment of the heart was first digested to isolate myocytes and non-myocytes and it was observed that ENPP1 expression was restricted to the non-myocyte fraction of cells. qPCR demonstrated that the expression of ENPP1 was almost 100-fold higher in non-myocytes compared to myocytes. Immunostaining of the injured region demonstrated that cardiomyocytes did not express ENPP1 and ENPP1 expressing cells in the injury region co-stained for the cardiac fibroblast marker vimentin (FIG. 1H). To confirm this observation, immunostaining in mice harboring genetically labeled cardiac fibroblasts was performed. For this purpose, Col1a2CreERT2 [17] mice or TCF21MerCreMer(MCM) [18] (TCF21 and Col1a2 being fibroblast Cre drivers) mice were crossed with the lineage reporter Rosa26tdTomato mice and administered tamoxifen for 10 days to 8-10 week old progeny mice. Mice were subsequently subjected to ischemic cardiac injury and immunostaining of injured heart sections demonstrated ENPP1 to be expressed by genetically labeled cardiac fibroblasts (FIGS. 1I, J). For an independent corroboration, flow cytometry was performed on the non-myocyte cells from the injured heart and observed that the fraction of ENPP1 expressing non-myocyte cells increased by greater than 2 fold compared to that in the uninjured region (15.9±1.3% in the uninjured region to 35.8±5% in the injured region, mean±S.E.M., n=8 *p<0.05). Following cardiac injury, 68%, 72% and 86% of ENPP1 expressing cells co-expressed the fibroblast markers CD90.2, TCF21MCM induced tdTomato label and MEFSK4 respectively. Macrophages and endothelial cells comprised the remaining fraction of non-myocyte cells expressing ENPP1. Finally, to confirm the distribution of ENPP1 across the non-myocyte population in the injured heart, single cell RNA-seq data sets of the non-myocyte fraction[15] isolated at 7 days after ischemic cardiac injury were analyzed and consistent with flow cytometry and immunostaining, observed that ENPP1 was primarily expressed by cardiac fibroblasts and to a lesser degree by macrophages and endothelial cells (FIG. 1K).

### Example 2: In Response to Increased Expression of ENPP1 by Non-Myocyte Cells, the Cardiac Muscle Cell Secretes Pro-Apoptotic Molecules that Cause Cell Death of Non-Myocytes

Extracellular ATP is a damage associated molecular pattern (DAMP) signal and extracellular ATP concentration is known to increase by several orders of magnitude after tissue injury[19]. As ENPP1 hydrolyzes extracellular ATP and is expressed after cardiac injury in non-myocytes but not in myocytes, it was hypothesized that ENPP1 via its enzymatic activity may be regulating intercellular communication between myocytes and non-myocytes in the injured region. To interrogate how ENPP1 mediates a cross talk between myocytes and non-myocytes, first cardiac fibroblasts were isolated by enzymatic digestion of the murine heart and stably over-expressed the mouse ENPP1 gene using a lentivirus. Cardiac fibroblasts were selected to over-express ENPP1 as ENPP1 is primarily expressed by cardiac fibroblasts in vivo after heart injury. To avoid culture induced senescence of cardiac fibroblasts and variation associated with primary cell isolation, the studies immortalized ENPP1 overexpressing cardiac fibroblasts by lentivirally introducing the SV40 antigen (ENPP1-CFs). Control cardiac fibroblasts (Control-CFs) infected with an empty lentivirus without the ENPP1 transgene were also immortalized in a similar manner. To determine the role of ENPP1 in mediating myocyte-non myocyte cross talk, ENPP1-CFs were co-cultured and Control CFs with neonatal rat ventricular cardiomyocytes (cardiac muscle cells) and then added ATP. Within 48 hours of incubation, it was observed there was a 75% reduction in the number of ENPP1 over-expressing cardiac fibroblasts with no effect on the numbers of cardiac muscle cells (FIGS. 2A, B). In the absence of exogenously added ATP, there was no reduction in ENPP1-CFs, and ATP did not significantly affect the cell numbers of Control-CFs co-cultured with rodent cardiomyocytes (FIG. 2B). In the absence of co-culture with cardiomyocytes, addition of ATP did not result in reduction of ENPP1-CFs (FIG. 2C). These observations strongly suggest that an interaction between ENPP1-CFs, ATP and cardiomyocytes was causing cell death of cardiac fibroblasts. Considering these observations, it was hypothesized that the combination of ENPP1 and ATP was inducing the cardiomyocytes to secrete molecules that were causing death of cardiac fibroblasts. To investigate this hypothesis, ATP was added and recombinant ENPP1 protein to rodent cardiomyocytes. Following 24 hours of incubation, the conditioned medium from these cardiomyocytes was collected and then treated cardiac fibroblasts (grown in a separate plate in the absence of any cardiomyocytes) with the myocyte conditioned medium so collected (FIG. 2D). Control conditioned medium included conditioned medium collected from cardiomyocytes in an identical manner after treatment with either vehicle, ENPP1 or ATP respectively. Within 48 hours of addition of ENPP1+ATP myocyte conditioned medium, cardiac fibroblasts underwent cell death (FIG. 2D). Propidium iodide (PI) and Annexin V staining with flow cytometry demonstrated a two to four-fold increase in cell death of cardiac fibroblasts treated with ENPP1+ATP myocyte conditioned medium compared to control conditioned medium (FIG. 2E). TUNEL staining and cleaved caspase 3 activity confirmed the apoptotic cell death of cardiac fibroblasts treated with ENPP1+ATP myocyte conditioned medium (31% and 18% of cardiac fibroblasts stained positive for TUNEL or Cleaved Caspase 3 staining respectively compared to 5% and 2% of cardiac fibroblasts treated with control myocyte conditioned medium, *p<0.01) (FIGS. 2F,G). To make sure that the immortalization process itself did not make the cardiac fibroblasts sensitive to myocyte conditioned medium, primary cardiac fibroblasts were isolated, treated with ENPP1+ATP myocyte conditioned medium and observed a similar degree of cell death. These observations strongly suggest that in response to recombinant ENPP1 protein and ATP, the cardiomyocytes secreted pro-apoptotic molecules that were inducing cell death of cardiac fibroblasts but not the myocytes themselves.

To determine whether the ability to secrete cell death inducing molecules in response to ENPP1 and ATP was a specific property of cardiomyocytes, ENPP1 recombinant protein and ATP was added to cardiac fibroblasts. Transfer of ENPP1+ATP CF conditioned medium to cardiac fibroblasts grown separately in another plate did not cause cell death. These observations thus show that the ability to secrete pro-apoptotic small molecules in the presence of ENPP1 and ATP is specific to cardiomyocytes. Next, it was investigated whether human cardiomyocytes exhibited this same property of secreting pro-apoptotic molecules following treatment with ATP and ENPP 1. cardiomyogenic differentiation of human pluripotent stem cells (hPSCs) were indcued, treated hPSC derived cardiomyocytes with ENPP1 and ATP , collected the conditioned medium, added it to human cardiac fibroblasts grown in a separate dish and observed a similar degree of cell death. These observations confirm the ability of human cardiomyocytes to respond to ENPP1 and ATP in a similar manner as rodent cardiomyocytes.

As ENPP1 is a transmembrane protein with an extracellular catalytic domain, it was next investigated whether the ectonucleotidase activity of ENPP1 was required for this interaction with cardiomyocytes to generate pro-apoptotic molecules. To determine this, an expression construct for a mutant ENPP1 (mutant ENPP1, containing a single amino acid substitution in the catalytic domain) was created [16], that is devoid of nucleotidase activity. The studies lentivirally overexpressed the mutant ENPP1 construct in cardiac fibroblasts and subsequently immortalized cardiac fibroblasts as previously stated (mutant ENPP1-CFs). When mutant ENPP1-CFs or control ENPP1-CFs were co-cultured with rodent cardiomyocytes, the addition of ATP induced cell death of ENPP1-CFs whereas minimal cell death was observed in mutant ENPP1-CFs. These experiments thus demonstrated that the catalytic domain of ENPP1 is necessary for the cardiomyocytes to generate pro-apoptotic molecules in the presence of extracellular ATP. ENPP1 hydrolyzes extracellular ATP directly into adenosine monophosphate (AMP) and pyrophosphate (PPi), and so if the catalytic domain of ENPP1 is necessary for the myocytes to generate pro-apoptotic molecules, it follows that either AMP or PPi alone should be able to reproduce the combined effect of ENPP1 and ATP on cardiomyocytes. To determine which hydrolytic product could act on the cardiomyocyte to generate pro-apoptotic molecules, AMP or PPi was added to cardiomyocytes, collected the conditioned medium after 24 hours and then added the conditioned medium to cardiac fibroblasts grown separately. It was observed that AMP treated myocyte conditioned medium caused cell death but not PPi treated myocyte conditioned medium (FIGS. 2H, I). However, the addition of AMP alone to cardiac fibroblasts did not induce cell death thus excluding a potential toxic effect of AMP alone on cardiac fibroblasts. These observations thus demonstrate that AMP, a product generated by ENPP1 mediated hydrolysis of ATP, induces the myocyte to generate pro-apoptotic molecules that cause cell death of cardiac fibroblasts.

As cell death of cardiac fibroblasts was occurring within 48 hours of treatment with ENPP1+ATP myocyte conditioned medium, it was next investigated the dynamics of cell death. To determine the dynamics of cell death, quantitative phase microscopy (QPM) was performed to determine the biomass of numerous individual cardiac fibroblasts following treatment with either control conditioned or ENPP1+ATP myocyte conditioned medium. As cells undergo apoptosis, the surface area and the cell biomass of the affected cells decreases [20]. It was observed that the cardiac fibroblasts treated with ENPP1+ATP myocyte conditioned medium exhibited an increasing cell biomass with stable cell surface area for the first 12 hours, similar to cardiac fibroblasts treated with control conditioned medium. After 12 hours, cardiac fibroblasts treated with ENPP1+ATP myocyte conditioned medium exhibited a rapid and significant decline in cell biomass and cell surface area due to apoptosis, which was not observed in the control condition. Plots of single cell surface area versus individual biomass clearly demonstrated significant mean differences in cell size and biomass of cardiac fibroblasts after 24 hours of treatment with ENPP1+ATP myocyte conditioned medium versus control conditioned medium.

In addition to cardiac fibroblasts, macrophages and endothelial cells also expressed ENPP1 after cardiac injury. It was next investigated whether the pro-apoptotic molecules released by cardiomyocytes in response to ENPP1 and ATP also induce cell death of other non-myocyte cells. Cardiomyocytes were treated with ENPP1 and ATP, collected the conditioned medium and added it to macrophages, endothelial or smooth muscle cells grown separately and observed that conditioned medium was able to induce cell death in these non-myocyte population as well (FIGS. 2J,K). However, ENPP1 and ATP myocyte conditioned medium did not induce cell death of cardiomyocytes grown separately (FIG. 2L). These observations demonstrate that in response to ENPP1+ATP, cardiomyocyte secreted pro-apoptotic molecules can cause cell death of a wide variety of non-myocyte cells but the myocyte itself remains immune to such death inducing molecules.

### Example 3: Genetic Deletion of ENPP1 Leads to Enhanced Cardiac Wound Healing after Ischemic Injury

Before determining the identity of the pro-apoptotic molecules secreted by myocytes in response to ENPP1+ATP, it was investigated the physiological role of ENPP1 in regulating cardiac wound healing in vivo. Cardiac fibroblasts, macrophages, endothelial cells and smooth muscle cells are known to play a vital role in cardiac repair after ischemic cardiac injury. Ablation of macrophages with the drug clodronate prior to myocardial infarction leads to dysregulated wound healing with left ventricular dilatation and development of cardiac dysfunction and loss of endocardial integrity[21, 22]. The angiogenic response of endothelial cells is known to promote neovascularization and post infarct cardiac recovery[23] and inactivation or deletion of cardiac fibroblasts can lead to compromised cardiac healing and development of heart failure or cardiac rupture[12, 24]. It was hypothesized that if increased ENPP1 expression in the heart promotes the cardiac muscle cell to secrete molecules that exert adverse effects on non-myocyte cells, then cardiac repair or wound healing should be augmented by inhibiting ENPP 1. The next metrics of augmented wound healing were defined. It is well established that superior wound healing should lead to better post injury organ function. Increasing inflammation has been known to be associated with worse cardiac wound healing outcomes[25] while increased angiogenesis is thought to be favorable for cardiac healing and post injury function [26]. Finally, as the heart heals with scar formation, increase in scar size is considered to be an adverse outcome of wound healing[15]. Thus, better preservation of post injury cardiac function, increased angiogenesis, decreased inflammation and decreased post injury scar size were defined by as quantifiable outcomes to characterize enhanced wound healing.

As ENPP1 was predominantly expressed by cardiac fibroblasts and ENPP1 expression significantly overlapped with the expression of Col1a2 in cardiac fibroblasts on single cell RNA-seq, it was used the Col1a2CreERT driver to conditionally delete ENPP1 in cardiac fibroblasts. The ENPP1asj/asj mutant mouse was not used as musculoskeletal problems in the globally mutant adult animals prevented from performing surgical procedures on the animals. The Col1a2CreERT animals were crossed with animals that had both ENPP1 alleles foxed (ENPP1fl/fl) [27] and progeny mice were administered tamoxifen from 5 days prior to cardiac injury to 7 days after to maximize ENPP1 deletion (ENPP1 conditional knockout or ENPP1CKO). Western blotting demonstrated that following injury, ENPP1 expression in the injured area increased by more than 5 fold in the injured region in control (Cre(−):ENPP1fl/fl animals) but there was no increase in ENPP1 expression in the injured ENPP1CKO hearts (FIGS. 3A,B). Animals were subjected to weekly echocardiography and ENPP1CKO animals demonstrated significantly better preservation of post injury organ function and decreased chamber size compared to control littermates (FIGS. 3C,D). Significantly beneficial effects on cardiac contractile function (ejection fraction or fractional shortening) and chamber dilatation were evident as early as 7 days after injury (EF of 52.74% in ENPP1CKO animals versus 32.05% in control littermates at 7 days following injury, n=14 in wild type littermates and n=16 in ENPP1CKO animals, **p<0.01) (FIG. 3D). Similarly, cardiac chamber size in both systole and diastole was significantly decreased in the ENPP1 CKO animals (LVIDs of 2.934 mm in ENPP1 CKO animals versus 3.855 mm in control littermates at 7 days following injury, n=14 in wild type littermates and n=16 in ENPP1 CKO animals, **p<0.01) (FIG. 3D). It was defined as mild, moderate or severe depression in post injury cardiac contractile function as EF>40%, between 20% and 40% and less than 20% respectively. Almost 50% of the control littermates exhibited severe depression in EF or heart failure at 7 days after injury but only 6% of the animals in the ENPP1CKO group exhibited severe depression in EF (FIG. 3E). The degree of fibrosis or scar size measured at 4 weeks post injury, both at the apical and mid ventricular regions of the heart was significantly lower in the ENPP1CKO animals (average fibrotic surface area of 27.65±4.19% of LV surface area in control littermates versus 11.85±2.96% in ENPP1CKO animals, n=14 in control and n=16 in ENPP1CKO animals, *p<0.05) (FIGS. 3F,G). Again, the scar size was classified as mild (<20% of LV surface area), moderate (20-40% of LV surface area and severe (>40% of LV surface area) and observed that approximately 21% of the control animals exhibited severe fibrosis at 4 weeks after MI in contrast to less than 6.2% in the ENPP1CKO animals (FIG. 3H). Post infarct hypertrophy is an adverse outcome of wound healing and it was observed that the heart weight/body weight ratio was significantly lower in the ENPP1CKO animals at 4 weeks post injury (no change in body weight alone) (FIG. 3I). Histology of the peri-infarct area confirmed significantly smaller myocyte surface area or decreased hypertrophy in ENPP1CKO hearts (FIG. 3J). Examination of capillary density by CD31 staining (canonical endothelial marker) demonstrated a significantly greater capillary count in the ENPP1CKO animals at 4 weeks after injury (FIG. 3K). These observations demonstrate that cardiac wound healing is significantly enhanced in ENPP1CKO animals with better preservation of post injury function, greater angiogenesis, significantly decreased scarring and significantly attenuated post injury myocyte hypertrophy.

### Example 4: Genetic Variation of ENPP1 in Heart Predicts Adverse Cardiac Outcomes Across 100 Diverse Strains of Mice

To further strengthen the observations on the role of ENPP1 in regulating cardiac wound healing in vivo, the studies employed a systems genetics approach using alternative models of cardiac injury. The hybrid mouse diversity panel comprises 100 diverse classical and recombinant inbred strains of mice which can be subjected to cardiac injury to identify genetic determinants of post injury cardiac traits [29, 30]. The mouse strains in the HMDP were treated with 3 weeks of continuous isoproterenol infusion that results in cardiomyocyte hypertrophy and interstitial fibrosis. In contrast to the ischemic cardiac injury model that is characterized by myocyte loss, chamber dilatation and replacement fibrosis, the isoproterenol model is characterized by a more chronic hypertrophic injury with interstitial fibrosis and chamber dilatation occurring at later stages[31]. Animals were followed by serial echocardiograms to determine ejection fraction and hearts harvested to determine LV gene expression changes. Gene expression signatures were statistically correlated with clinical traits to identify significant relationships across all the strains. Using this system, it was initially observed a large degree of genetic variation in ENPP1 expression, particularly following isoproterenol infusion. Next, it was determined whether this genetic regulation of expression of ENPP1 in the heart significantly correlated with post injury cardiac traits of cardiac contractility, chamber size and fibrosis. It was observed that ENPP1 expression significantly correlated with the development of adverse post injury traits such as LV hypertrophy (cardiac mass), chamber size, decreased cardiac contractility and degree of fibrosis following isoproterenol infusion. As a control, the studies examined a post injury cardiac phenotype unrelated to wound healing such as heart rate and did not see any significant correlation with ENPP1 expression. These observations using systems genetics approaches and alternative injury models thus provide compelling evidence that ENPP1 is a strong driver of cardiac repair and support the principal hypothesis that ENPP1 regulates cardiac wound healing.

### Example 5: Single Cell RNA-Seq of ENPPICKO Animals Post Ischemic Injury Demonstrates Downregulation of Pro-Inflammatory, Apoptotic and Fibrotic Pathways

The ENPP1 genetic loss of function data along with the systems genetics approaches provide compelling evidence that loss of ENPP1 is associated with better functional cardiac outcomes after organ injury. It has been hypothesized that increased activity of the ENPP1/ATP axis in the injured region promotes pro-death pathways in non-myocytes. To further investigate this hypothesis and to determine changes in transcriptional signatures of non-myocyte cells in the heart after cardiac injury, the studies performed single cell RNA-seq on control and ENPP1CKO hearts at 7 days following injury. The studies subjected ENPP1CKO and littermate control animals to ischemic cardiac injury, isolated the non-myocyte fraction and subjected the cells to single cell RNA-seq using the 10× genomics platform. It was observed fibroblasts, macrophages and endothelial cells as the largest contributors to the non-myocyte population. The studies next determined the distribution of the ENPP1CKO and control wild type genotypes in the non-myocyte cell population and observed increased numbers of endothelial cells and decreased numbers of macrophages in the ENPP1CKO animals (consistent with increased angiogenesis and decreased inflammation). As ENPP1 had been deleted in Col1a2 expressing cardiac fibroblasts it was specifically examined the cardiac fibroblast population and first confirmed decrease in ENPP1 expression. The number of myofibroblasts (myofibroblasts represent activated fibroblasts identified by Acta2 expression) that secrete extracellular matrix and form scar tissue was decreased in ENPP1CKO hearts. Other markers of activated myofibroblasts such as Cnn2 (calponin) and Tagln (transgelin)[15] were also decreased in ENPP1CKO cardiac fibroblasts suggestive of an attenuation of a scarring response. A gene ontology analysis of genes differentially expressed in fibroblasts demonstrated downregulation of extracellular matrix (ECM) organization, inflammatory and apoptotic pathways. Canonical genes known to regulate ECM deposition were significantly downregulated in cardiac fibroblasts in ENPP1CKO animals. Analysis of apoptotic pathways demonstrated downregulation of pro-apoptotic genes or genes inducing growth arrest and upregulation of anti-apoptotic genes in cardiac fibroblasts of ENPP1CKO animals compared to those of control littermates. Transcriptomic signatures of macrophages were also consistent with decreased expression of pro-inflammatory genes in macrophages in hearts of ENPP1CKO animals. Histology at 7 days post injury showed decreased collagen deposition along with a significantly decreased number of macrophages and increased number of capillaries, findings consistent with the RNA-seq analysis. These observations demonstrate that genetic deletion of ENPP1 switches the wound healing transcriptional response after cardiac injury to a more pro-reparative one with less inflammation, less scarring and greater angiogenesis.

### Example 6: Cardiomyocyte Secreted Metabolites Rather Than Proteins Cause Cell Death of Non-Myocytes

The genetic loss of function experiments along with the hybrid mouse diversity panel experiments strongly suggest that ENPP1 regulates cardiac wound healing in vivo. It was next investigated mechanisms of action of ENPP1. The data demonstrates that in the presence of ENPP1 and ATP, cardiomyocytes secrete pro-apoptotic factors that cause cell death of non-myocytes in vitro and that loss of ENPP1 in vivo is associated with down-regulation of pro-apoptotic pathways in non-myocytes. To obtain insight into the mechanisms of ENPP1 in regulating myocyte-non myocyte cross talk, the studies next sought to identify the pro-apoptotic molecules secreted by cardiomyocytes in response to the presence of ATP and ENPP 1. It was first determined whether the pro-apoptotic molecules were proteins or metabolites. For this purpose, the studies collected the myocyte conditioned medium following addition of ENPP1 and ATP and subjected it to high heat (95° C.) for 15 minutes to enable denaturation of proteins. When added to cardiac fibroblasts, the heat-treated conditioned medium retained biological activity and induced cardiac fibroblast cell death. The degree of cell death was similar to that induced by ENPP1+ATP myocyte conditioned medium not subjected to heat inactivation. These results suggest that the pro-apoptotic molecules are likely to be heat stable metabolites rather than proteins that are denatured by heat. To confirm these results that a metabolite and not a protein was likely mediating pro-apoptotic effects, the studies next passed the ENPP1+ATP myocyte conditioned medium through a protein fractionation column with a filter cutoff of 3 kilo Daltons (kD) and then treated cardiac fibroblasts with the protein rich (>3 kD) or protein poor fractions (<3 kD) of the conditioned medium. The protein rich fraction (MW>3 kD) did not cause cell death but the conditioned medium filtrate less than 3 kD induced cardiac fibroblast cell death. These observations taken together suggest that metabolites rather than proteins secreted by cardiomyocytes are causing cell death of non-myocyte cells.

To determine the identity of the metabolites, the studies collected myocyte conditioned medium following treatment of the myocytes with ENPP1, ATP, ENPP1+ATP, AMP or PPi and subjected the conditioned medium to LC-MS analysis. The studies identified metabolites that were differentially present between ENPP1+ATP or AMP treated myocyte conditioned medium versus ENPP1, ATP or PPi treated myocyte conditioned medium. These metabolites mainly related to purine/pyrimidine biosynthesis/catabolism pathways and did not include any known pro-apoptotic factors. The studies treated cardiac fibroblasts with each of the top 7 most differentially upregulated metabolites in the ENPP1+ATP or AMP myocyte conditioned but none of these metabolites caused cell death. It was hypothesized that the metabolite causing cell death might be present at very low concentration or alternatively a combination of metabolites that was not evident might be needed for cell death.

### Example 7: Death of Non-Myocytes is Related to Cell Proliferation

Unable to readily identity the pro-apoptotic metabolites, the studies sought alternative physiologic principles of approaching the problem. The data has consistently shown that the ENPP1+ATP myocyte conditioned medium caused death of a wide range of non-myocyte cells, but that myocytes themselves are not affected. This could be possibly secondary to a specific receptor that is present in non-myocytes and not in myocytes. However, as non-myocyte cells of different lineages are affected, it was hypothesized that the ability of myocytes to be immune to the conditioned medium likely reflects an inherent cellular property of myocytes that distinguishes itself from that in non-myocyte cells tested by us. As non-myocytes are proliferative and myocytes are non-proliferative, it was hypothesized that the ability to cycle was making the non-myocytes susceptible to the metabolites secreted by the myocyte. To determine whether this was true, the studies harvested primary cardiac fibroblasts and induced cell cycle arrest of freshly harvested cardiac fibroblasts by irradiating them. It was observed that the ENPP1+ATP myocyte conditioned medium induced cell death of primary cardiac fibroblasts that were not subject to irradiation but was unable to induce cell death in irradiated cardiac fibroblasts (FIGS. 4A,B). To confirm this finding, the studies next treated cardiac fibroblasts with the cell cycle inhibitor mitomycin C and again observed cell cycle arrested cardiac fibroblasts were resistant to cell death induced by ENPP1+ATP myocyte conditioned medium (FIGS. 4C,D). To obtain an independent confirmation on the cell cycle dependent cell death of cardiac fibroblasts treated with ENPP1+ATP myocyte conditioned medium, the studies repeated the experiments with primary mouse embryonic fibroblasts (mEF) and confirmed that cell cycle arrest with irradiation or mitomycin C prevented cell death of mEF (FIGS. 4E-H). These observations suggest that cell cycling of the target non-myocyte cells is necessary for the myocyte secreted molecules to cause cell death.

The studies next examined the mechanisms of cell death secondary to cell cycling. First, the studies performed RNA-seq on cardiac fibroblasts treated with ENPP1+ATP, AMP, ENPP1, ATP or vehicle treated myocyte conditioned medium. Principal component analysis at 24 and 48 hours demonstrated that the gene expression signatures of cardiac fibroblasts treated with ENPP1+ATP or AMP myocyte conditioned medium were similar and clearly distinguishable from those of the other groups (FIG. 4I). A gene ontogeny analysis demonstrated significant upregulation of the p53 signaling pathway (FIG. 4J) with significant upregulation of p53 regulated pro-apoptotic genes (FIG. 4K) in cardiac fibroblasts treated with ENPP1+ATP or AMP myocyte conditioned medium. p53 is known to regulate cell cycle arrest and drive a DNA damage response causing cell death so the studies next examined in detail the phases of cell cycle that were disrupted in non-myocytes treated with ENPP1+ATP myocyte conditioned medium. For this purpose, the studies treated cardiac fibroblasts with ENPP1+ATP treated myocyte conditioned medium for 48 hours and performed PI staining and flow cytometry to determine the phase of cell cycle that was affected. At 48 hours there was clear evidence of Gl/S phase arrest with decrease in the number of cardiac fibroblasts in G1 and an increase in the number of cells in the sub G1 phase (FIGS. 4L,M). Western blotting demonstrated significant upregulation of DNA damage response markers (Gamma H2A.X) and the checkpoint kinase 1 (pCHK1) that is known to regulate a DNA damage response and cell cycle arrest (FIG. 4N). It was hypothesized that p53 was driving the DNA damage response and mediating apoptotic death. Phosphorylation of serinel5 in p53 has been shown to initiate DNA damage response[32, 33] and the studies observed increased phosphorylation of p53Ser15 in cardiac fibroblasts treated with ENPP1+ATP myocyte conditioned medium (FIG. 4O). To determine whether a p53 initiated DNA damage response was required for cell death, the studies deleted the p53 gene in cardiac fibroblasts by infecting primary cardiac fibroblasts isolated from p53 floxed mice[34] with a lentiviral Cre in vitro. Prior to infecting with the Cre lentivirus, the fibroblasts (from hearts of p53 foxed mice) were immortalized with SV40 to maintain experimental consistency. Deletion of p53 was confirmed with Western blotting (FIG. 4P) and the studies observed that cardiac fibroblasts lacking p53 in contrast to wild type controls were resistant to ENPP1+ATP myocyte conditioned medium induced cell death (FIGS. 4Q,R). These experiments thus demonstrate that metabolite/s secreted by the cardiac muscle cell in response to ENPP1 and ATP initiate a p53 dependent DNA damage response and apoptosis in cycling non-myocyte cells.

### Example 8: Myocyte Secreted Metabolite(s) Disrupt Pyrimidine Biosynthesis in Cycling Non-Myocytes to Cause Cell Death

The experiments related to the ability of ENPP1+ATP treated myocyte conditioned medium to initiate a DNA damage response in cycling non-myocyte cells strongly suggested that the metabolite(s) interfere with the cell cycle machinery. It also provided an obvious explanation as to why non-proliferative myocytes are immune to the effects of the metabolite(s). A nucleotide balance between the content of purines and pyrimidines available to cycling cells is critical to avoid genotoxic stress and maintain genomic stability [35]. It was hypothesized that disruption of the nucleotide biosynthetic pathways in non-myocyte cells and an imbalance of purine versus pyrimidine nucleotides was inducing genotoxic stress and initiating a DNA damage response in proliferating non-myocytes. The studies treated cardiac fibroblasts with ENPP1+ATP myocyte conditioned medium for 24 hours and measured the content of nucleoside monophosphate and nucleoside triphosphates in treated cardiac fibroblasts by LC/MS-MS. Consistent with the hypothesis, the studies observed that the pyrimidines cytidine and uridine mono and triphosphates (CMP, CTP, UMP, UTP) were significantly reduced in cardiac fibroblasts treated with ENPP1+ATP myocyte conditioned medium compared to cardiac fibroblasts treated with vehicle myocyte conditioned medium (FIG. 5A) while purine nucleotide levels were slightly increased or remained unaltered (FIG. 5B). These observations suggested that defects in pyrimidine biosynthesis resulting in insufficient pyrimidine precursors were likely leading to a DNA damage response in cycling non-myocytes and resulting in cell death. To determine whether this is true, the studies attempted to rescue cell death of cardiac fibroblasts by adding uridine or deoxycytidine to the ENPP1+ATP myocyte conditioned medium. Cardiac fibroblasts were treated with the ENPP1+ATP myocyte conditioned medium but uridine or deoxycytidine or both were added to the cardiac fibroblasts at the time of addition of ENPP1+ATP myocyte conditioned medium (FIG. 5C). The studies observed that addition of uridine or deoxycytidine or both completely rescued cell death (FIGS. 5D,E). Deoxycytidine serves as a precursor of dCTP synthesis via the enzyme deoxycytidine kinase[36]. When the studies added a specific inhibitor of deoxycytidine kinase (DI-87)[37], deoxycytidine was unable to prevent cell death thereby strongly supporting the hypothesis that reduced availability of pyrimidines was causing cell death (Fig SF,G).

### Example 9: Inhibition of UMP Synthase Step is the Underlying Cause of Defects in Pyrimidine Biosynthesis

Pyrimidine biosynthesis occurs via a sequence of well-regulated steps (FIG. 5H), where carbamoyl phosphate is converted to carbamoyl aspartate and then to dihydroorotate. Dihydroorotate dehydrogenase (DHODH) then converts dihydroorotate to the pyrimidine orotate. In the presence of phosphoribosyl pyrophosphate (PRPP), orotate is converted by phosphoribosyl transferase activity of UMP synthase to orotidine monophosphate (OMP) and then decarboxylated by OMP decarboxylase activity of UMP synthase to uridine monophosphate (UMP). To determine which steps in pyrimidine biosynthesis are affected in cycling non-myocytes, the studies treated cardiac fibroblasts with ENPP1+ATP myocyte conditioned medium and 24 hours later harvested the cells and subjected the cardiac fibroblasts to mass spectrometry to determine metabolites in the pyrimidine biosynthesis pathway. Cardiac fibroblasts treated with ENPP1+ATP myocyte conditioned medium compared to vehicle treated myocyte conditioned medium showed significantly increased amounts of carbamoyl aspartate, dihydroorotate and orotate but decreased orotidine, uridine, UMP, UDP, UTP as well as CTP (FIG. 5I). This suggests that the early steps of pyrimidine biosynthesis are not affected, but the ENPP1+ATP myocyte conditioned medium is inhibiting later steps of uridine monophosphate synthesis in cardiac fibroblasts. As orotate levels were increased but orotidine levels decreased in cardiac fibroblasts treated with ENPP1+ATP myocyte conditioned medium, it was hypothesized that inhibition was occurring at the OMP synthesis step from orotate and PRPP (orotate phosphoribosyl transferase). To determine this, the studies added OMP to cardiac fibroblasts at the time of addition of ENPP1+ATP myocyte conditioned medium. OMP completely rescued cell death (FIGS. 5J,K) thereby strongly suggesting that the ENPP1+ATP myocyte conditioned medium inhibits pyrimidine biosynthesis at the OMP synthesis step in cycling non-myocytes. Finally, to demonstrate that defects in pyrimidine biosynthesis are sufficient to cause cell death, the studies treated cardiac fibroblasts with a specific inhibitor of DHODH (brequinar) and observed cardiac fibroblast cell death thereby demonstrating that disruption in pyrimidine biosynthesis is sufficient to cause cell death in proliferating cardiac fibroblasts (FIGS. 5L,M).

### Example 8: Adenine is a Critical Metabolite Secreted by Cardiomyocytes in Response to Increased ENPP1 and ATP that is Necessary for Causing Cell Death in Non-Myocytes

The data so far suggests that a metabolite or metabolites secreted by cardiomyocytes in the presence of ENPP1 and ATP induce defects in pyrimidine biosynthesis of cycling non-myocytes and initiate a p53 mediated DNA damage response that results in cell death of non-myocytes. With this in mind, the studies returned to the central question of the identity of such pro-apoptotic metabolites. As the metabolites were inhibiting pyrimidine biosynthesis, it was hypothesized that the metabolites were likely nucleotides or their derivatives or compounds that inhibited specific enzymes in pyrimidine biosynthesis. To filter potential candidates differentially present in the ENPP1+ATP myocyte conditioned medium, the studies performed high performance liquid chromatography (HPLC) to determine physico-chemical properties of the candidates. The studies performed HPLC by passing the ENPP1+ATP myocyte conditioned medium through a CN (cyano-propyl) solid phase chromatography column that retains polar compounds to determine whether the metabolites inducing cell death were predominantly polar or non-polar. The hydrophobic flow through as well as the hydrophilic retentate following elution were vacuum dried, reconstituted and added to cardiac fibroblasts and the studies observed that the hydrophobic fraction caused cell death, but the hydrophilic eluate did not. To confirm this, the studies next passed the ENPP1+ATP myocyte conditioned medium through a C18 stationary phase chromatography column which retains non-polar compounds on the column and the polar fraction flows through. In agreement with the observations made with the CN column, the studies observed that the hydrophobic fraction eluted by acetonitrile (ACN) induced cell death but hydrophilic flow through did not.

The chromatography experiments suggested that the cell death inducing metabolites in the ENPP1+ATP MCndM were retained by the C18 column and eluted by acetonitrile (ACN). To further narrow down potential candidates based on their retention and ACN elution properties, the studies next increased the resolution of HPLC and joined two reverse phase HPLC columns in series, loaded ENPP1+ATP myocyte conditioned medium onto the columns and used a linear gradient of ACN to collect 80 fractions eluted with increasing concentrations of ACN. Pools of 10 fractions were vacuum dried, reconstituted, and added to cardiac fibroblasts and the studies observed that fractions 41-50 from ENPP1+ATP myocyte conditioned medium (corresponding approximately to ACN concentrations between 40 and 50%) reliably resulted in cardiac fibroblast cell death, while those of control myocyte conditioned medium did not. To confirm these observations, the studies loaded the C18 column with ENPP1+ATP myocyte conditioned medium and eluted fractions at 5%, 25%, 50%, 75%, and 100% ACN, which were vacuum dried, reconstituted, and added to cardiac fibroblasts. Cell death was only observed with the 50% ACN fraction (FIG. 6A). The studies then subjected the 50% ACN eluates of ENPP1+ATP and control conditioned medium to LC-MS analysis, focusing on nucleosides, nucleotides, and their derivatives. The studies cross checked this list with the mass spectrometry data on the unfractionated ENPP1+ATP conditioned medium to ensure that the compounds were present in the unfractionated ENPP1+ATP myocyte conditioned medium. The studies chose 7 compounds which were highly enriched in the 50% ACN elutes of the ENPP1+ATP myocyte treated conditioned medium (FIG. 6B). Addition of all 7 compounds to cardiac fibroblasts caused severe cell death (FIGS. 6C,D). As uridine supplementation had rescued cell death of cardiac fibroblasts treated with the unfractionated ENPP1+ATP myocyte conditioned medium, the studies added uridine together with the 7 compounds to cardiac fibroblasts and observed that cell death was prevented (FIGA. 6C,D). This suggested that the mechanism of cell death following addition of the 7 selected compounds was similar to that mediated by ENPP1+ATP myocyte conditioned medium. Moreover, as these compounds were present in high abundance in ENPP1+ATP myocyte conditioned medium as well as in the specific ACN eluate that retained biological activity, it was likely that this set contained the mediator of cell death of non-myocytes. To determine which combination of compounds induced cell death, the studies subtracted each compound one by one from the set of 7 compounds to determine which compound was necessary for cell death (FIG. 6E). The studies observed that adenine was critically necessary for cell death as removal of adenine from the 7 compounds resulted in no cell death (FIGS. 6E,F). Removal of any one of the other molecules did not affect or reduce cell death (FIGS. 6E,F). The studies next determined whether adenine was sufficient for cell death of non-myocytes. When cardiac fibroblasts were treated with adenine alone, no cell death was observed, demonstrating that adenine though necessary was not sufficient for cell death (FIGS. 6G,H). The studies next added adenine plus one of the other 6 compounds and observed that addition of adenine with either adenosine, inosine or IMP (inosine monophosphate) or AMP was sufficient to cause death (FIGS. 6G,H). Combinations of adenine with either hypoxanthine, xanthine or orotate did not cause cell death (FIGS. 6G,H). These observations thus demonstrate adenine in combination with specific purine nucleosides can induce non-myocyte cell death. Next, the studies wanted to confirm that the combination of adenine and adenosine induced arrest in pyrimidine biosynthesis and thus cell death could be rescued by addition of OMP or uridine. When cardiac fibroblasts were treated with both adenine and adenosine plus either OMP or uridine, the studies observed rescue of cell death (FIGS. 6I,J). This thus demonstrates that the combination of adenine and a purine nucleoside, both present in the ENPP1+ATP myocyte conditioned medium, is sufficient to cause disruption of pyrimidine biosynthesis and induce cell death of non-myocytes and that such cell death can be rescued with pyrimidine supplementation. The studies also added adenine and adenosine to macrophages, endothelial cells and smooth muscle cells and noted cell death, demonstrating that a combination of adenine and adenosine could induce cell death on a wide variety of non-myocyte cells.

Next, the studies wanted to determine whether adenine was a key critical component of the ENPP1+ATP myocyte conditioned medium that induced cell death of non-myocytes. The studies adopted a loss of function approach to determine whether catabolic removal of adenine would rescue ENPP1+ATP myocyte conditioned medium from causing cell death of cardiac fibroblasts. There is no mammalian enzyme that catabolizes adenine but plants and express adenine deaminase which converts adenine to hypoxanthine[39]. The studies lenti-virally expressed yeast adenine deaminase (also known as adenine amino hydrolase, AAH) in cardiac fibroblasts and immortalized them to create a stable cell line. Cardiac fibroblasts expressing adenine deaminase were resistant to cell death induced by ENPP1+ATP myocyte conditioned medium compared to control GFP expressing cardiac fibroblasts (FIGS. 6K,L). These experiments thus conclusively demonstrate that adenine is a key molecule secreted by cardiomyocytes in response to ENPP1 and ATP that is necessary for cell death of non-myocytes. Considering these observations on the toxicity of adenine in combination with adenosine or other specific purine nucleosides and the rescue effect of uridine, the studies checked the 25%, 50% and 75% ACN eluates of the ENPP1+ATP myocyte conditioned medium passed through the C18 column for the levels of adenine, adenosine, IMP, and uridine. Uridine and IMP eluted much earlier so that the ratio of adenine+adenosine/uridine or adenosine+IMP/uridine was much greater in the 50% than the 25% or 75% ACN eluate, suggesting the potential use of the adenine+adenosine or adenosine+IMP to uridine ratios as a cytotoxic metric.

The data suggests that pyrimidine synthesis is disrupted at the OMP synthesis step. The studies next investigated potential reasons for the inhibition of OMP synthesis. Phosphoribosyl pyrophosphate (PRPP) is critically required for cell cycle completion[40]. For pyrimidine biosynthesis, PRPP is the donor of phospho-ribose groups for OMP synthesis from orotate as well as in the purine salvage pathway to synthesize AMP from adenine. PRPP synthesis by PRPP synthetase is potently inhibited by AMP and ADP [41]. The model illustrated in the findings suggest that the toxicity of the combination of adenine and adenosine could be related to inhibition of PRPP synthetase by AMP with concomitant consumption of PRPP by adenine catalyzed by adenine phosphoribosyl transferase. If so, PRPP levels should be significantly reduced and the studies observed significantly decreased PRPP levels in cardiac fibroblasts treated with ENPP1+ATP myocyte conditioned medium (FIG. 6M) along with decreased levels of metabolites that are generated using PRPP as a substrate such as NAD with corresponding increase in nicotinamide (PRPP is required by the nicotinamide salvage pathway to convert nicotinamide into its mono nucleotide). AMP and not adenosine induces the cardiomyocyte to generate adenine The studies have shown here that the catalytic domain of ENPP1 is essential for its interaction with ATP and cardiomyocytes to generate pro-apoptotic molecules. AMP a metabolite generated by the hydrolytic activity of ENPP1 on ATP was able to induce the myocyte to secrete pro-apoptotic metabolites. Having identified that adenine is a critical mediator of non-myocyte cell death, the studies next investigated whether AMP or its metabolite adenosine is needed for the myocyte to secrete pro-apoptotic molecules. Extracellular AMP is hydrolyzed by CD73, a membrane bound protein, to form adenosine[42]. To determine whether adenosine is mediating effects of AMP on the myocyte, the studies added AMP to cardiomyocytes in the presence or absence of a CD73 inhibitor, collected the conditioned medium and subsequently added it to cardiac fibroblasts. The studies observed that inhibition of CD73 (using two different inhibitors, AB680, AMP-CP)[43, 44] significantly increases cardiac fibroblast cell death, strongly suggesting that AMP and not adenosine is necessary for the cardiomyocytes to secrete pro-apoptotic molecules. Indeed, conditioned medium from adenosine treated cardiomyocytes was unable to induce cell death of cardiac fibroblasts. Moreover, myocyte conditioned medium collected after the addition of adenosine receptor agonist NECA[45] did not cause fibroblast cell death. The studies added ENPP1+ATP to cardiomyocytes in the presence of adenosine receptor antagonists [46-48] and did not observe any change in the ability of ENPP1+ATP myocyte conditioned medium to cause death of cardiac fibroblasts. This was performed to provide another piece of evidence that adenosine, a product of AMP hydrolysis was not inducing the cardiomyocyte to secrete pro-apoptotic molecules. Finally, to demonstrate the critical role of AMP, the studies added an adenosine kinase inhibitor (ABT-702) [49 ] or AMP deaminase inhibitor (cpd3) [50 ] to cardiomyocytes at the time of addition of AMP. Adenosine kinase inhibitors would decrease the generation of AMP from adenosine, while AMP deaminase inhibitors would increase AMP concentrations by inhibiting AMP deamination. The studies observed that adenosine kinase inhibitors attenuated cell death of cardiac fibroblasts while AMP deaminase inhibitors worsened cell death of cardiac fibroblasts demonstrating that AMP was critically required by the cardiomyocytes to generate pro-apoptotic molecules.

As adenine was found to be a critical molecule secreted by the cardiomyocyte that was necessary for non-myocyte cell death, the studies investigated whether AMP was directly utilized by the cardiac muscle cell for adenine synthesis. For this purpose, the studies added ENPP1+ N15 labeled ATP to cardiomyocytes, collected the conditioned medium and then treated cardiac fibroblasts with the conditioned medium so collected. The studies then harvested cardiomyocytes treated with ENPP1+N15 labeled ATP, the conditioned medium as well as cardiac fibroblasts to determine the fraction of adenine and other key metabolites that would bear the isotope label. The fraction of N15 labeled adenine in the cardiomyocytes was 77% of the total adenine present, while 98% of the adenine in the conditioned medium and 82% of the adenine in the cardiac fibroblasts was labeled. The labeled adenine in the cardiomyocytes contained five 15N atoms, demonstrating direct conversion of 15N5 AMP to adenine by the cardiomyocyte. Also, the majority of the adenine in the conditioned medium had all five nitrogen atoms labeled, demonstrating that adenine synthesized by the cardiomyocyte directly from AMP is the predominant source of adenine in the conditioned medium. Similarly, the majority of the adenosine, IMP, inosine, and AMP in the cardiomyocyte, conditioned medium, as well as the cardiac fibroblasts was labeled. The fraction of labeled nucleoside guanosine was much lower in the cardiomyocyte, while in the cardiac fibroblasts almost 60% of guanosine was labeled. All four nitrogens in the purine ring of guanosine were 15N, suggesting it was derived from the 15N5 AMP. Labeling of unrelated metabolites such as glutamate, not typically derived from adenosine derivatives was expectedly low demonstrating the fidelity of the system as a negative labeling example.

Taken together these observations demonstrate that in the presence of ENPP1, ATP is hydrolyzed to form AMP which is then metabolized by the cardiac muscle cell to form adenine and other purine nucleosides that are then secreted/released by the myocyte into the extracellular environment. Both metabolites are then taken up by proliferating non-myocytes to exert biological effects.

### Example 9: Uridine Administration after Heart Injury Augments Cardiac Repair and Function in Vivo

Uridine supplementation to non-myocytes treated with ENPP1+ATP myocyte conditioned medium rescued cell death. It was hypothesized that if ENPP1 worsened cardiac repair by inducing defects in pyrimidine biosynthesis in vivo then administration of uridine following ischemic cardiac injury should rescue pyrimidine biosynthesis, augment wound healing and lead to better preservation of post injury cardiac function.

The studies subjected wild type C57BL/6 animals to ischemic cardiac injury and administered uridine by continuous infusion for 14 days starting from the day of injury and measured cardiac function weekly for 4 weeks (FIG. 7A). Echocardiography demonstrated that uridine significantly preserved post injury cardiac contractile function (ejection fraction and fractional shortening) compared to vehicle injected animals (EF of 37.02±3.12% in uridine group versus 23.92±3.36% in vehicle group, FS of 18.01±1.68% in uridine group versus 11.24±1.70% in vehicle group, n=15/vehicle and n=15/uridine groups, **p<0.01 at day 7 after injury) with a trend towards better preservation of end systolic ventricular diameters (FIGS. 7B,C). The studies defined mild, moderate and severe depression in EF as EF>40%, between 20 and 40% and less than 20%. The studies observed that 71% of the vehicle injected animals exhibited severe depression in EF compared to only 13% of the animals which received uridine (FIG. 7D). Histology demonstrated decreased fibrosis measured at the apex and mid ventricle at 4 weeks post injury in uridine injected animals (FIGS. 7E,F). The studies again stratified the degree of post infarct fibrosis as mild (<20% of LV surface area), moderate (20-40%) and severe (>40% of LV surface area) and observed that approximately 43% of the animals in the vehicle group exhibited severe post infarct fibrosis but none of the animals that received uridine exhibited severe fibrosis (FIG. 7G). Hearts of uridine injected animals exhibited decreased heart weight/body weight ratios compared to vehicle injected animals suggestive of attenuation of adverse ventricular hypertrophy (FIG. 7H). There was significantly increased capillary density in uridine treated hearts at 4 weeks following injury (FIG. 7I). These observations demonstrate that supplementation of the pyrimidine uridine is sufficient to augment cardiac wound healing in vivo with significantly better cardiac contractile function, decreased scarring and augmented angiogenesis. These data taken together provide proof of concept that pyrimidine supplementation represents a therapeutic strategy for heart repair.

### Example 10: Screening of Small Molecule Libraries to Identify ENPP1 Inhibitors as Therapeutic Agents to Augment Wound Healing After Ischemic Cardiac Injury

The data using both in vitro and in vivo systems has provided evidence that ENPP1, upregulated at the region of injury, contributes to worsened wound healing and worsening post injury cardiac function. Considering these observations, it was hypothesized that ENPP1 could serve as a therapeutic target for augmenting cardiac wound healing following ischemic injury. To identify small molecule inhibitors of ENPP1, the studies established a cell free luciferase based luminescent assay and screened a large small molecule library comprising more than 200,000 compounds available at the institution. This assay is based on the principle that when ATP is incubated with luciferase, light is generated that can detected by a luminometer. When ENPP1 protein is added, ATP is hydrolyzed and thus the degree of luminescence declines. Next when a small molecule ENPP1 inhibitor is also added, then nucleotidase activity of ENPP1 is inhibited so greater amount of residual ATP leads to a stronger signal. Thus, hits can be identified as ENPP1 inhibitors by a gain of signal. The studies filtered the hits to select compounds that have been used previously in humans or animals to avoid issues associated with toxic effects of untested compounds and to facilitate repurposing of known drugs. Myricetin, a polyphenolic flavonoid[51], was the leading hit with almost 99% inhibition of ENPP1 enzymatic activity and an IC50 of 4.8 μM. The studies next tested the ability of myricetin to prevent non-myocyte cell death in vitro. The studies co-cultured ENPP1 over-expressing cardiac fibroblasts with rodent ventricular cardiomyocytes. Addition of ATP led to increased cell death of cardiac fibroblasts but concomitant addition of myricetin (10 uM) significantly attenuated cell death. Next, the studies treated cardiomyocytes with ENPP1+ATP and myricetin, collected the conditioned medium and added it to cardiac fibroblasts and observed a significant reduction in cell death with the ENPP1+ATP+myricetin myocyte conditioned medium compared to ENPP1+ATP myocyte conditioned medium.

Subsequently, the studies determined whether administration of myricetin can augment cardiac wound healing in vivo and lead to better preservation of post injury heart function. The studies subjected C57BL/6 animals to ischemic cardiac injury and administered vehicle or 30 mg/kg myricetin intra-peritoneally to the animals starting on the day of injury and continuing daily for 14 days post injury (FIG. 8A). To confirm that myricetin inhibited ENPP1 activity in the injured hearts of these animals, the studies harvested the injured hearts following 7 days of myricetin administration and observed significant increase in ATP hydrolytic activity post injury in the vehicle treated group but animals treated with myricetin did not demonstrate a significant increase in ATP hydrolytic ability, demonstrating the ability of myricetin to inhibit cardiac ENPP1 activity in vivo at the dose used (FIG. 8B). Serial echocardiography demonstrated significantly better preservation of post injury contractile function and significantly decreased chamber size compared to vehicle injected animals reminiscent of the genetic loss of function data shown earlier.

At day 7 after injury, in the myricetin injected group versus the vehicle injected group, the studies measured ejection fraction of 38.04±3.74% versus 15.40±3.47% and fractional shortening of 18.67±2.05% versus 7.07±1.64% (n=12 in vehicle and n=15 in myricetin treated animals, **p<0.01). Ventricular chamber size demonstrated a LVIDs of 3.68±0.23 in myricetin treated group versus 4.75±0.31 in vehicle treated group, n=12 in vehicle and n=15 in myricetin treated animals, **p<0.01) (FIGS. 8C,D). More than 66% of the animals in the vehicle injected group had severe heart failure (defined here as EF<20%) while only 23% of the animals receiving myricetin developed severe heart failure (FIG. 8E). The area of scarring measured at the mid ventricular and apical regions at 4 weeks post injury was also significantly reduced in myricetin treated animals (FIGS. 8F,G). The fraction of animals with severe fibrosis (defined as >40% of the LV surface area) was approximately 56% in the vehicle treated animals but was reduced to 15% in the myricetin treated group (FIG. 8H). Myricetin treated animals exhibited decreased ventricular hypertrophy and had significantly lower heart weight/body weight ratios without any change in body weight. Myricetin treated animals also demonstrated a significant increase in capillary density compared to the vehicle treated groups.

Arrest in pyrimidine biosynthesis in non-myocyte cells treated with ENPP1+ATP myocyte conditioned medium initiated a DNA damage response with expression of gamma H2A.X and p53Ser15 phosphorylation. The studies performed immunostaining and observed attenuated expression of gamma H2A.X and p53Ser15 phosphorylation in non-myocyte cells in hearts of myricetin treated animals (FIGS. 8I,J). Taken together, these observations are consistent with the hypothesis of an ENPP1 driven p53 mediated DNA damage response initiated by purine/pyrimidine imbalance and its attenuation by inhibition of ENPP1. Finally, as inhibition of ENPP1 should rescue pyrimidine biosynthesis, the studies performed metabolomic analysis of the hearts of wild type and myricetin treated animals after cardiac injury. The studies observed that animals that received myricetin exhibited significant increase in levels of the pyrimidines, uridine and cytidine in the injured regions of the heart and exhibited decrease in carbamoyl phosphate levels compared to vehicle treated injured hearts (FIG. 8K). As uridine rescues the toxic effects of the combination of adenine and a purine nucleoside, the studies measure the adenine+adenosine/uridine ratio in cardiac tissue as a metric of cytoxicity and observed that hearts of animals treated with myricetin exhibited a decreased cytotoxicity ratio (FIG. 8L). The studies also performed metabolomic analysis of the serum to determine whether circulating pyrimidine metabolites could serve as biomarkers of effective therapy. The studies observed orotate to decrease in the serum of myricetin treated animals, while deoxyuridine and orotidine levels increased consistent with rescue of pyrimidine biosynthesis (FIG. 8M). These in vivo data remarkably mirror the in vitro experiments demonstrating an arrest in pyrimidine biosynthesis at the OMP synthesis step and suggesting the potential use of serum orotidine as a biomarker to monitor the therapy or prognosis of wound healing in the heart.

The observations demonstrate a hitherto unappreciated role of the cardiac muscle cell in modulating pyrimidine biosynthesis of non-muscle cells by releasing extracellular adenine and specific nucleosides. Such paracrine mechanisms of metabolic control are especially germane after cardiac injury where extracellular ATP released from necrotic myocytes is hydrolyzed by the ectonucleotidase ENPP1 to form AMP. AMP serves as a key stress response signal to the myocyte and induces the myocyte to synthesize adenine base and specific ribonucleosides that are then secreted/released into the extracellular environment to exert cytotoxic effects on proliferating non-myocytes. The isotope labeling experiments suggest that AMP is directly used by the myocyte to generate adenine, such as by nucleotide phosphorolysis. Ribonucleosides such as inosine or IMP also exhibit high isotope labeling suggestive of direct conversion of labeled AMP to inosine or IMP. The combination of adenine and specific ribonucleosides such as adenosine or inosine exert cytotoxic effects on proliferating non-myocytes by disrupting their pyrimidine biosynthesis. Decreased availability of uridine and cytidine creates a purine/pyrimidine imbalance within the proliferating non-myocytes, induces genotoxic stress and initiates a p53 mediated DNA damage response causing cell cycle arrest. The imbalance of purines/pyrimidines is a key event initiating the cell cycle arrest/apoptotic cascade as supplementation of uridine to correct decreased pyrimidine levels rescues cell death. The studies demonstrate that disruption of pyrimidine biosynthesis occurs at the step of OMP synthesis from orotate and PRPP by UMP synthase, which is likely secondary to low PRPP levels in the proliferating non-myocytes. PRPP is also required for purine biosynthesis either via the de novo or salvage pathway but purine nucleotide levels are likely spared because AMP can be converted into GMP via intermediates of IMP and XMP without the need for PRPP. The labeling data supports this conclusion since 60% of the guanosine of fibroblasts treated with ENPP1+ATP myocyte conditioned medium contained a purine ring with four 15N atoms that was derived from the 15N5 labeled AMP. As cardiomyocytes have exited the cell cycle, they thus remain immune to the purine/pyrimidine imbalance on cell cycle and the linked DNA damage response. In this regard, the data highlights the differential effects of extracellular adenine on cells based on their cycling status.

The data suggests an inherent defect in cardiac repair based on the ability of non-proliferative cardiac muscle cells to mount a ‘metabolic attack’ and a DNA damage response in proliferative non-myocyte cells that play a critical role in wound healing. The teleological reasons why a cardiac muscle cell would mount such a repair response that ultimately is counterproductive for cardiac healing remain unclear, but such mechanisms of metabolic control could have evolved as a defense response against rapidly proliferating non cardiac cells as in an invading cancer or microbes. Genotoxic stress and an associated DNA damage response in non-myocytes likely results in non-myocyte cellular dysfunction and loss of mechanisms regulating angiogenesis, inflammation and extracellular matrix production. Pharmacological inhibition of the ENPP1/AMP cascade increases pyrimidine biosynthesis in the heart and attenuates the DNA damage response in non-myocyte cells and leads to augmented wound healing with greater angiogenesis, decreased inflammation and diminished scar size. The disruption of pyrimidine biosynthesis occurs at the OMP synthesis step in vitro and pharmacological targeting of ENPP1 in vivo results in increased serum orotidine levels suggesting a rescue of OMP synthesis and the potential of using serum orotidine as a biomarker for monitoring disruption in pyrimidine biosynthesis and its successful targeting during tissue repair. In summary, the observations taken together, demonstrate a model of tissue repair where an inter-cellular regulation of pyrimidine biosynthesis determines how the heart heals itself.

### REFERENCES

1. Miyazawa, H. and A. Aulehla. Revisiting the role of metabolism during development. Development. 2018. 145(19).
2. Jones, R. G., D. R. Plas, S. Kubek, M. Buzzai, J. Mu, Y. Xu, M. J. Birnbaum, and C. B. Thompson. AMP-activated protein kinase induces a p53-dependent metabolic checkpoint. Mol Cell. 2005. 18(3): p. 283-93.
3. Machler, P., M. T. Wyss, M. Elsayed, J. Stobart, R. Gutierrez, A. von Faber-Castell, V. Kaelin, M. Zuend, A. San Martin, I. Romero-Gomez, F. Baeza-Lehnert, S. Lengacher, B. L. Schneider, P. Aebischer, P. J. Magistretti, L. F. Barros, and B. Weber. In Vivo Evidence for a Lactate Gradient from Astrocytes to Neurons. Cell Metab. 2016. 23(1): p. 94-102.
4. Nahrendorf, M., F. K. Swirski, E. Aikawa, L. Stangenberg, T. Wurdinger, J. L. Figueiredo, P. Libby, R. Weissleder, and M. J. Pittet. The healing myocardium sequentially mobilizes two monocyte subsets with divergent and complementary functions. J Exp Med. 2007. 204(12): p. 3037-47.
5. Frangogiannis, N. G. Emerging roles for macrophages in cardiac injury: cytoprotection, repair, and regeneration. J Clin Invest. 2015. 125(8): p. 2927-30.
6. Frangogiannis, N. G. The extracellular matrix in myocardial injury, repair, and remodeling. J Clin Invest. 2017. 127(5): p. 1600-1612.
7. Coade, S. B. and J. D. Pearson. Metabolism of adenine nucleotides in human blood. Circ Res. 1989. 65(3): p. 531-7.
8. Trautmann, A. Extracellular ATP in the immune system: more than just a “danger signal”. Sci Signal. 2009. 2(56): p. pe6.
9. Burnstock, G. Purinergic Signaling in the Cardiovascular System. Circ Res. 2017. 120(1): p. 207-228.
10. Venereau, E., C. Ceriotti, and M. E. Bianchi. DAMPs from Cell Death to New Life. Front Immunol. 2015. 6: p. 422.
11. Kato, K., H. Nishimasu, E. Mihara, R. Ishitani, J. Takagi, J. Aoki, and O. Nureki. Expression, purification, crystallization and preliminary X-ray crystallographic analysis of Enpp1. Acta Crystallogr Sect F Struct Biol Cryst Commun. 2012. 68(Pt 7): p. 778-82.
12. Kanisicak, O., H. Khalil, M. J. Ivey, J. Karch, B. D. Maliken, R. N. Correll, M. J. Brody, J. L. SC, B. J. Aronow, M. D. Tallquist, and J. D. Molkentin. Genetic lineage tracing defines myofibroblast origin and function in the injured heart. Nat Commun. 2016. 7: p. 12260.
13. Masse, K., S. Bhamra, G. Allsop, N. Dale, and E. A. Jones. Ectophosphodiesterase/nucleotide phosphohydrolase (Enpp) nucleotidases: cloning, conservation and developmental restriction. Int J Dev Biol. 2010. 54(1): p. 181-93.
14. Moller, S., C. Jung, S. Adriouch, G. Dubberke, F. Seyfried, M. Seman, F. Haag, and F. Koch-Nolte. Monitoring the expression of purinoceptors and nucleotide-metabolizing ecto-enzymes with antibodies directed against proteins in native conformation. Purinergic Signal. 2007. 3(4): p. 359-66.
15. Yokota, T., J. McCourt, F. Ma, S. Ren, S. Li, T. H. Kim, Y. Z. Kurmangaliyev, R. Nasiri, S. Ahadian, T. Nguyen, X. H. M. Tan, Y. Zhou, R. Wu, A. Rodriguez, W. Cohn, Y. Wang, J. Whitelegge, S. Ryazantsev, A. Khademhosseini, M. A. Teitell, P. Y. Chiou, D. E. Birk, A. C. Rowat, R. H. Crosbie, M. Pellegrini, M. Seldin, A. J. Lusis, and A. Deb. Type V Collagen in Scar Tissue Regulates the Size of Scar after Heart Injury. Cell. 2020.
16. Li, Q., H. Guo, D. W. Chou, A. Berndt, J. P. Sundberg, and J. Uitto. Mutant Enpp1asj mice as a model for generalized arterial calcification of infancy. Dis Model Mech. 2013. 6(5): p. 1227-35.
17. Ubil, E., J. Duan, I. C. Pillai, M. Rosa-Garrido, Y. Wu, F. Bargiacchi, Y. Lu, S. Stanbouly, J. Huang, M. Rojas, T. M. Vondriska, E. Stefani, and A. Deb. Mesenchymal-endothelial transition contributes to cardiac neovascularization. Nature. 2014. 514(7524): p. 585-90.
18. Acharya, A., S. T. Baek, S. Banfi, B. Eskiocak, and M. D. Tallquist. Efficient inducible Cre-mediated recombination in Tcf21 cell lineages in the heart and kidney. Genesis. 2011. 49(11): p. 870-7.
19. Corriden, R. and P. A. Insel. Basal release of ATP: an autocrine paracrine mechanism for cell regulation. Sci Signal. 2010. 3(104): p. rel.
20. Zangle, T. A., D. Burnes, C. Mathis, O. N. Witte, and M. A. Teitell. Quantifying biomass changes of single CD 8+ T cells during antigen specific cytotoxicity. PLoS One. 2013. 8(7): p. e68916.
21. Frantz, S., U. Hofmann, D. Fraccarollo, A. Schafer, S. Kranepuhl, I. Hagedorn, B. Nieswandt, M. Nahrendorf, H. Wagner, B. Bayer, C. Pachel, M. P. Schon, S. Kneitz, T. Bobinger, F. Weidemann, G. Ertl, and J. Bauersachs. Monocytes/macrophages prevent healing defects and left ventricular thrombus formation after myocardial infarction. FASEB J. 2013. 27(3): p. 871-81.
22. van Amerongen, M. J., M. C. Harmsen, N. van Rooij en, A. H. Petersen, and M. J. van Luyn. Macrophage depletion impairs wound healing and increases left ventricular remodeling after myocardial injury in mice. Am J Pathol. 2007. 170(3): p. 818-29.
23. Cochain, C., K. M. Channon, and J. S. Silvestre. Angiogenesis in the infarcted myocardium. Antioxid Redox Signal. 2013. 18(9): p. 1100-13.
24. Duan, J., C. Gherghe, D. Liu, E. Hamlett, L. Srikantha, L. Rodgers, J. N. Regan, M. Rojas, M. Willis, A. Leask, M. Majesky, and A. Deb. Wnt1/betacatenin injury response activates the epicardium and cardiac fibroblasts to promote cardiac repair. EMBO J. 2012. 31(2): p. 429-42.
25. Ong, S. B., S. Hernandez-Resendiz, G. E. Crespo-Avilan, R. T. Mukhametshina, X. Y. Kwek, H. A. Cabrera-Fuentes, and D. J. Hausenloy. Inflammation following acute myocardial infarction: Multiple players, dynamic roles, and novel therapeutic opportunities. Pharmacol Ther. 2018. 186: p. 73-87.
26. Kobayashi, K., K. Maeda, M. Takefuji, R. Kikuchi, Y. Morishita, M. Hirashima, and T. Murohara. Dynamics of angiogenesis in ischemic areas of the infarcted heart. Scientific Reports. 2017. 7(1): p. 7156.
27. Roberts F L, Rashdan A N, Phadwal K, Markby G R, Dillon S R, Zoll J, Berger J, Milne E, Orriss I R, Karsenty G, Le Saux O, Morton NM, Farquharson C, and M. V E. Osteoblast-specific deficiency of ectonucleotide pyrophosphatase phosphodiesterase-1 engenders insulin resistance in high-fat diet fed mice. Journal of Cell Physiology. 2020 (In press).
28. Schuleri, K. H., M. Centola, K. S. Evers, A. Zviman, R. Evers, J. A. C. Lima, and A. C. Lardo. Cardiovascular magnetic resonance characterization of peri-infarct zone remodeling following myocardial infarction. Journal of Cardiovascular Magnetic Resonance. 2012. 14(1): p. 24.
29. Rau, C. D., A. J. Lusis, and Y. Wang. Systems Genetics for Mechanistic Discovery in Heart Diseases. Circ Res. 2020. 126(12): p. 1795-1815.
30. Rau, C. D., J. Wang, R. Avetisyan, M. Romay, L. Martin, S. Ren, Y. Wang, and A. J. Lusis. Mapping Genetic Contributions to Cardiac Pathology Induced by Beta-Adrenergic Stimulation in Mice. Circ Cardiovasc Genet. 2014.
31. Wang, J. J., C. Rau, R. Avetisyan, S. Ren, M. C. Romay, G. Stolin, K. W. Gong, Y. Wang, and A. J. Lusis. Genetic Dissection of Cardiac Remodeling in an Isoproterenol-Induced Heart Failure Mouse Model. PLoS Genet. 2016. 12(7): p. e1006038.
32. Nghiem, P., P. K. Park, Y. S. Kim Ys, B. N. Desai, and S. L. Schreiber. ATR is not required for p53 activation but synergizes with p53 in the replication checkpoint. J Biol Chem. 2002. 277(6): p. 4428-34.
33. Canman, C. E., D. S. Lim, K. A. Cimprich, Y. Taya, K. Tamai, K. Sakaguchi, E. Appella, M. B. Kastan, and J. D. Siliciano. Activation of the ATM kinase by ionizing radiation and phosphorylation of p53. Science. 1998. 281(5383): p. 1677-9.
34. Marino, S., M. Vooijs, H. van Der Gulden, J. Jonkers, and A. Berns. Induction of medulloblastomas in p53-null mutant mice by somatic inactivation of Rb in the external granular layer cells of the cerebellum. Genes Dev. 2000. 14(8): p. 994-1004.
35. Pai, C. C. and S. E. Kearsey. A Critical Balance: dNTPs and the Maintenance of Genome Stability. Genes (Basel). 2017. 8(2).
36. Hazra, S., S. Ort, M. Konrad, and A. Lavie. Structural and kinetic characterization of human deoxycytidine kinase variants able to phosphorylate 5-substituted deoxycytidine and thymidine analogues. Biochemistry. 2010. 49(31): p. 6784-90.
37. Poddar, S., E. V. Capparelli, E. W. Rosser, R. M. Gipson, L. Wei, T. Le, M. E. Jung, C. Radu, and M. Nikanjam. Development and preclinical pharmacology of a novel dCK inhibitor, DI-87. Biochem Pharmacol. 2020. 172: p. 113742.
38. Sykes, D. B., Y. S. Kfoury, F. E. Mercier, M. J. Wawer, J. M. Law, M. K. Haynes, T. A. Lewis, A. Schajnovitz, E. Jain, D. Lee, H. Meyer, K. A. Pierce, N. J. Tolliday, A. Waller, S. J. Ferrara, A. L. Eheim, D. Stoeckigt, K. L. Maxey, J. M. Cobert, J. Bachand, B. A. Szekely, S. Mukherjee, L. A. Sklar, J. D. Kotz, C. B. Clish, R. I. Sadreyev, P. A. Clemons, A. Janzer, S. L. Schreiber, and D. T. Scadden. Inhibition of Dihydroorotate Dehydrogenase Overcomes Differentiation Blockade in Acute Myeloid Leukemia. Cell. 2016. 167(1): p. 171-186 e15.
39. Deeley, M. C. Adenine deaminase and adenine utilization in Saccharomyces cerevisiae. J Bacteriol. 1992. 174(10): p. 3102-10.
40. Fridman, A., A. Saha, A. Chan, D. E. Casteel, R. B. Pilz, and G. R. Boss. Cell cycle regulation of purine synthesis by phosphoribosyl pyrophosphate and inorganic phosphate. Biochem J. 2013. 454(1): p. 91-9.
41. Switzer, R. L. and D. C. Sogin. Regulation and mechanism of phosphoribosylpyrophosphate synthetase. V. Inhibition by end products and regulation by adenosine diphosphate. J Biol Chem. 1973. 248(3): p. 1063-73.
42. Allard, B., M. S. Longhi, S. C. Robson, and J. Stagg. The ectonucleotidases CD39 and CD73: Novel checkpoint inhibitor targets. Immunol Rev. 2017. 276(1): p. 121-144.
43. Lawson, K. V., J. Kalisiak, E. A. Lindsey, E. T. Newcomb, M. R. Leleti, L. Debien, B. R. Rosen, D. H. Miles, E.U. Sharif, J. L. Jeffrey, J. B. L. Tan, A. Chen, S. Zhao, G. Xu, L. Fu, L. Jin, T. W. Park, W. Berry, S. Moschutz, E. Scaletti, N. Strater, N. P. Walker, S. W. Young, M. J. Walters, U. Schindler, and J. P. Powers. Discovery of AB680: A Potent and Selective Inhibitor of CD73. J Med Chem. 2020. 63(20): p. 11448-11468.
44. Knapp, K., M. Zebisch, J. Pippel, A. El-Tayeb, C. E. Muller, and N. Strater. Crystal structure of the human ecto-5′-nucleotidase (CD73): insights into the regulation of purinergic signaling. Structure. 2012. 20(12): p. 2161-73.
45. Lebon, G., T. Warne, P. C. Edwards, K. Bennett, C. J. Langmead, A. G. Leslie, and C. G. Tate. Agonist-bound adenosine A2A receptor structures reveal common features of GPCR activation. Nature. 2011. 474(7352): p. 521-5.
46. Glukhova, A., D. M. Thal, A. T. Nguyen, E. A. Vecchio, M. Jörg, P. J. Scammells, L. T. May, P. M. Sexton, and A. Christopoulos. Structure of the Adenosine A(1) Receptor Reveals the Basis for Subtype Selectivity. Cell. 2017. 168(5): p. 867-877.e13.
47. Jacobson, K. A., O. Nikodijević, W. L. Padgett, C. Gallo-Rodriguez, M. Maillard, and J. W. Daly. 8-(3-Chlorostyryl)caffeine (CSC) is a selective A2-adenosine antagonist in vitro and in vivo. FEBS Lett. 1993. 323(1-2): p. 141-4.
48. Brackett, L. E. and J. W. Daly. Functional characterization of the Alb adenosine receptor in NIH 3T3 fibroblasts. Biochem Pharmacol. 1994. 47(5): p. 801-14.
49. Annes, J. P., J. H. Ryu, K. Lam, P. J. Carolan, K. Utz, J. Hollister-Lock, A. C. Arvanites, L. L. Rubin, G. Weir, and D. A. Melton. Adenosine kinase inhibition selectively promotes rodent and porcine islet beta-cell replication. Proc Natl Acad Sci U S A. 2012. 109(10): p. 3915-20.
50. Admyre, T., L. Amrot-Fors, M. Andersson, M. Bauer, M. Bjursell, T. Drmota, S. Hallen, J. Hartleib-Geschwindner, B. Lindmark, J. Liu, L. Lofgren, M. Rohman, N. Selmi, and K. Wallenius. Inhibition of AMP deaminase activity does not improve glucose control in rodent models of insulin resistance or diabetes. Chem Biol. 2014. 21(11): p. 1486-96.
51. Ong, K. C. and H. E. Khoo. Biological effects of myricetin. Gen Pharmacol. 1997. 29(2): p. 121-6.

### INCORPORATION BY REFERENCE

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize or be able to ascertain using no more than routine experimentation, many equivalents to the specific embodiments of the invention described herein. Such equivalents are intended to be encompassed by the following claims.

What is claimed:

1. A method of monitoring ENPP1 activity after cardiac injury in a subject comprising determining whether a level of a pyrimidine nucleotide in serum of the subject is below a threshold level, wherein a level of the pyrimidine nucleotide below the threshold level is indicative of ENPP1 activity.

2. The method of claim 1, wherein the pyrimidine nucleotide is involved in pyrimidine biosynthesis.

3. The method of claim 1 or 2, wherein the pyrimidine nucleotide prevents cardiac cell death.

4. The method of any one of claims 1-3, wherein determining whether the level of the pyrimidine nucleotide is below a threshold level comprises measuring the level of the pyrimidine nucleotide in the serum.

5. The method of any one of claims 1-4, wherein the pyrimidine nucleotide is uridine.

6. The method of any one of claims 1-4, wherein the pyrimidine nucleotide is uridine monophosphate (UMP).

7. The method of any one of claims 1-4, wherein the pyrimidine nucleotide is uridine triphosphate (UTP).

8. The method of any one of claims 1-4, wherein the pyrimidine nucleotide is cytidine.

9. The method of any one of claims 1-4, wherein the pyrimidine nucleotide is cytidine monophosphate (CMP).

10. The method of any one of claims 1-4, wherein the pyrimidine nucleotide is cytidine triphosphate (CTP).

11. The method of any one of claims 1-4, wherein the pyrimidine nucleotide is orotate.

12. The method of any one of claims 1-4, wherein the pyrimidine nucleotide is deoxyuridine.

13. The method of any one of claims 1-4, wherein the pyrimidine nucleotide is orotidine.

14. A method of monitoring ENPP1 activity after cardiac injury in a subject, comprising:
(a) determining a level of a purine nucleotide and/or a purine nucleoside in serum of the subject;
(b) determining a level of a pyrimidine nucleotide in serum of the subject; and
(c) determining whether a ratio of the level of the purine nucleotide and/or the purine nucleoside to the level of the pyrimidine nucleotide is above a threshold level, wherein a ratio above the threshold level is indicative of ENPP1 activity.

15. The method of claim 14, wherein the purine nucleotide and/or the purine nucleoside disrupts pyrimidine biosynthesis.

16. The method of claim 14 or 15, wherein the purine nucleotide and/or the purine nucleoside induces cardiac cell death.

17. The method of any one of claims 14-16, wherein the pyrimidine nucleotide is involved in pyrimidine biosynthesis.

18. The method of any one of claims 14-17, wherein the pyrimidine nucleotide prevents cardiac cell death.

19. The method of any one of claims 14-18, wherein determining a level of a purine nucleotide and/or a purine nucleoside in serum of the subject comprises measuring the level of the purine nucleotide and/or purine nucleoside of the serum.

20. The method of any one of claims 14-19, wherein determining a level of a pyrimidine in serum of the subject comprises measuring the level of the pyrimidine nucleotide of the serum.

21. The method of any one of claims 14-20, wherein the purine nucleotide is adenine.

22. The method of any one of claims 14-20, wherein the purine nucleoside is adenosine.

23. The method of any one of claims 14-20, wherein the purine nucleoside is adenosine monophosphate (AMP).

24. The method of any one of claims 14-20, wherein the purine nucleoside is inosine.

25. The method of any one of claims 14-20, wherein the purine nucleoside is inosine monophosphate (IMP).

26. The method of any one of claims 14-25, wherein the pyrimidine nucleotide is uridine.

27. The method of any one of claims 14-25, wherein the pyrimidine nucleotide is uridine monophosphate (UMP).

28. The method of any one of claims 14-25, wherein the pyrimidine nucleotide is uridine triphosphate (UTP).

29. The method of any one of claims 14-25, wherein the pyrimidine nucleotide is cytidine.

30. The method of any one of claims 14-25, wherein the pyrimidine nucleotide is cytidine monophosphate (CMP).

31. The method of any one of claims 14-25, wherein the pyrimidine nucleotide is cytidine triphosphate (CTP).

32. The method of any one of claims 14-25, wherein the pyrimidine nucleotide is orotate.

33. The method of any one of claims 14-25, wherein the pyrimidine nucleotide is deoxyuridine.

34. The method of any one of claims 14-25, wherein the pyrimidine nucleotide is orotidine.

35. A method of treating myocardial infarction, promoting cardiac wound healing, enhancing cardiac repair, inhibiting ENPP1 activity, and/or preventing heart failure, cardiac cell death, ectopic calcification of cardiac tissue, scarring of cardiac tissue, dilated cardiomyopathy, and/or release of one or more pro-inflammatory molecules from cardiac myocytes in a subject, comprising:
(a) determining whether serum of the subject comprises a level of a pyrimidine nucleotide below a threshold level; and
(b) if the serum is characterized by a level below the threshold level, administering the pyrimidine nucleotide to the subject.

36. The method of claim 35, wherein the pyrimidine nucleotide is involved in pyrimidine biosynthesis.

37. The method of claim 35 or 36, wherein the pyrimidine nucleotide prevents cardiac cell death.

38. The method of any one of claims 35-37, wherein determining whether serum of the subject comprises a level of a pyrimidine nucleotide below a threshold level comprises measuring the level of the pyrimidine nucleotide of the serum.

39. The method of any one of claims 35-38, wherein the pyrimidine nucleotide is uridine.

40. The method of any one of claims 35-38, wherein the pyrimidine nucleotide is uridine monophosphate (UMP).

41. The method of any one of claims 35-38, wherein the pyrimidine nucleotide is uridine triphosphate (UTP).

42. The method of any one of claims 35-38, wherein the pyrimidine nucleotide is cytidine.

43. The method of any one of claims 35-38, wherein the pyrimidine nucleotide is cytidine monophosphate (CMP).

44. The method of any one of claims 35-38, wherein the pyrimidine nucleotide is cytidine triphosphate (CTP).

45. The method of any one of claims 35-38, wherein the pyrimidine nucleotide is orotate.

46. The method of any one of claims 35-38, wherein the pyrimidine nucleotide is deoxyuridine.

47. The method of any one of claims 35-38, wherein the pyrimidine nucleotide is orotidine.

48. The method of any one of claims 35-48, further comprising conjointly administering the pyrimidine nucleotide with an additional pyrimidine nucleotide to the subject.

49. The method of any one of claims 35-48, further comprising conjointly administering the pyrimidine nucleotide with an ENPP1 inhibitor to the subject.

50. The method of claim 49, whereby the pyrimidine nucleotide enhances the effects of the ENPP1 inhibitor relative to the ENPP1 inhibitor alone.

51. The method of claim 49 or 50, wherein the ENPP1 inhibitor is myricetin.

52. The method of any one of claims 35-51, further comprising conjointly administering the pyrimidine nucleotide and an adenosine kinase inhibitor to the subject.

53. The method of claim 52, whereby the pyrimidine nucleotide enhances the effects of the adenosine kinase inhibitor relative to the adenosine kinase inhibitor alone.

54. The method of any one of claims 35-53, further comprising conjointly administering the pyrimidine nucleotide and an adenosine monophosphate deaminase inhibitor to the subject.

55. The method of claim 54, whereby the pyrimidine nucleotide enhances the effects of the adenosine monophosphate deaminase inhibitor relative to the adenosine monophosphate deaminase inhibitor alone.

56. The method of any one of claims 35-55, further comprising conjointly administering the pyrimidine nucleotide and an additional cardiovascular therapeutic agent to the subject.

57. The method of claim 56, whereby the pyrimidine nucleotide enhances the effects of the additional cardiovascular therapeutic agent relative to the additional cardiovascular therapeutic agent alone.

58. A method of treating myocardial infarction, promoting cardiac wound healing, enhancing cardiac repair, inhibiting ENPP1 activity, and/or preventing heart failure, cardiac cell death, ectopic calcification of cardiac tissue, scarring of cardiac tissue, dilated cardiomyopathy, and/or release of one or more pro-inflammatory molecules from cardiac myocytes in a subject, comprising:
(a) determining a level of a purine nucleotide and/or a purine nucleoside in serum of the subject;
(b) determining a level of a pyrimidine nucleotide in serum of the subject;
(c) determining whether a ratio of the level of the purine nucleotide and/or the purine nucleoside to the level of the pyrimidine nucleotide is above a threshold level; and
(d) if the ratio of the level of the purine nucleotide and/or the purine nucleoside to the level of the pyrimidine nucleotide is above a threshold level, administering the pyrimidine nucleotide to the subject.

59. The method of claim 58, wherein the purine nucleotide and/or the purine nucleoside disrupts pyrimidine biosynthesis.

60. The method of claim 58 or 59, wherein the purine nucleotide and/or the purine nucleoside induces cardiac cell death.

61. The method of any one of claims 58-60, wherein the pyrimidine nucleotide is involved in pyrimidine biosynthesis.

62. The method of any one of claims 58-61, wherein the pyrimidine nucleotide prevents cardiac cell death.

63. The method of any one of claims 58-62, wherein determining a level of a purine nucleotide and/or a purine nucleoside in serum of the subject comprises measuring the level of the purine nucleotide and/or purine nucleoside of the serum.

64. The method of any one of claims 58-63, wherein determining a level of a pyrimidine in serum of the subject comprises measuring the level of the pyrimidine nucleotide of the serum.

65. The method of any one of claims 58-64, wherein the purine nucleotide is adenine.

66. The method of any one of claims 58-64, wherein the purine nucleoside is adenosine.

67. The method of any one of claims 58-64, wherein the purine nucleoside is adenosine monophosphate (AMP).

68. The method of any one of claims 58-64, wherein the purine nucleoside is inosine.

69. The method of any one of claims 58-64, wherein the purine nucleoside is inosine monophosphate (IMP).

70. The method of any one of claims 58-69, wherein the pyrimidine nucleotide is uridine.

71. The method of any one of claims 58-69, wherein the pyrimidine nucleotide is uridine monophosphate (UMP).

72. The method of any one of claims 58-69, wherein the pyrimidine nucleotide is uridine triphosphate (UTP).

73. The method of any one of claims 58-69, wherein the pyrimidine nucleotide is cytidine.

74. The method of any one of claims 58-69, wherein the pyrimidine nucleotide is cytidine monophosphate (CMP).

75. The method of any one of claims 58-69, wherein the pyrimidine nucleotide is cytidine triphosphate (CTP).

76. The method of any one of claims 58-69, wherein the pyrimidine nucleotide is orotate.

77. The method of any one of claims 58-69, wherein the pyrimidine nucleotide is deoxyuridine.

78. The method of any one of claims 58-69, wherein the pyrimidine nucleotide is orotidine.

79. The method of any one of claims 58-78, further comprising conjointly administering the pyrimidine nucleotide with an additional pyrimidine nucleotide to the subject.

80. The method of any one of claims 59-79, further comprising conjointly administering the pyrimidine nucleotide with an ENPP1 inhibitor to the subject.

81. The method of claim 80, whereby the pyrimidine nucleotide enhances the effects of the ENPP1 inhibitor relative to the ENPP1 inhibitor alone.

82. The method of claim 80 or 81, wherein the ENPP1 inhibitor is myricetin.

83. The method of any one of claims 58-82, further comprising conjointly administering the pyrimidine nucleotide and an adenosine kinase inhibitor to the subject.

84. The method of claim 83, whereby the pyrimidine nucleotide enhances the effects of the adenosine kinase inhibitor relative to the adenosine kinase inhibitor alone.

85. The method of any one of claims 58-84, further comprising conjointly administering the pyrimidine nucleotide and an adenosine monophosphate deaminase inhibitor to the subject.

86. The method of claim 85, whereby the pyrimidine nucleotide enhances the effects of the adenosine monophosphate deaminase inhibitor relative to the adenosine monophosphate deaminase inhibitor alone.

87. The method of any one of claims 58-86, further comprising conjointly administering the pyrimidine nucleotide and an additional cardiovascular therapeutic agent to the subject.

88. The method of claim 87, whereby the pyrimidine nucleotide enhances the effects of the additional cardiovascular therapeutic agent relative to the additional cardiovascular therapeutic agent alone.

89. A method of identifying a candidate ENPP1 inhibitor, comprising:
(a) contacting a cell sample with a test agent;
(b) measuring a level of a pyrimidine nucleotide of the cell sample; and
(c) identifying the test agent as a candidate ENPP1 inhibitor if the level of the pyrimidine nucleotide is increased as compared to a level of the pyrimidine nucleotide of a cell sample not contacted with the test agent.

90. The method of claim 89, wherein the level of the pyrimidine nucleotide of the cell sample not contacted with the test agent is the level of the pyrimidine nucleotide in the cell sample prior to contact with the test agent.

91. The method of claim 89 or 90, wherein the level of the pyrimidine nucleotide of the cell sample not contacted with the test agent is the level of the pyrimidine nucleotide of a corresponding control cell sample.

92. The method of any one of claims 89-91, wherein the pyrimidine nucleotide is involved in pyrimidine biosynthesis.

93. The method of any one of claims 89-92, wherein the pyrimidine nucleotide prevents cardiac cell death.

94. The method of any one of claims 89-93, wherein the pyrimidine nucleotide is uridine.

95. The method of any one of claims 89-93, wherein the pyrimidine nucleotide is uridine monophosphate (UMP).

96. The method of any one of claims 89-93, wherein the pyrimidine nucleotide is uridine triphosphate (UTP).

97. The method of any one of claims 989-93, wherein the pyrimidine nucleotide is cytidine.

98. The method of any one of claims 89-93, wherein the pyrimidine nucleotide is cytidine monophosphate (CMP).

99. The method of any one of claims 89-93, wherein the pyrimidine nucleotide is cytidine triphosphate (CTP).

100. The method of any one of claims 89-93, wherein the pyrimidine nucleotide is orotate.

101. The method of any one of claims 89-93, wherein the pyrimidine nucleotide is deoxyuridine.

102. The method of any one of claims 89-93, wherein the pyrimidine nucleotide is orotidine.

103. The method of any one of claims 89-102, further comprising measuring a level of cell death in the cell sample contacted with the test agent and determining if the level of cell death is decreased as compared to a level of cell death of the cell sample not contacted with the test agent.

104. A method of identifying a candidate ENPP1 inhibitor, comprising:
(a) contacting a cell sample with a test agent;
(b) measuring a level of a purine nucleotide of the sample;
(c) measuring a level of a pyrimidine nucleotide of the cell sample;
(d) determining a ratio of the level of the purine nucleotide to the level of the pyrimidine nucleotide; and
(e) identifying the test agent as a candidate ENPP1 inhibitor if the ratio is decreased as compared to a ratio of the level of the purine nucleotide to the level of the pyrimidine nucleotide of a cell sample not contacted with the test agent.

105. The method of claim 104, wherein the level of the purine nucleotide and the level of the pyrimidine nucleotide of the cell sample not contacted with the test agent is the level of the purine nucleotide and the level of the pyrimidine nucleotide in the cell sample prior to contact with the test agent.

106. The method of claim 104 or 105, wherein the level the purine nucleotide and the level of the pyrimidine nucleotide of the cell sample not contacted with the test agent is the level of the purine nucleotide and the level of the pyrimidine nucleotide of a corresponding control cell sample.

107. The method of any one of claims 104-106, wherein the purine nucleotide and/or the purine nucleoside disrupts pyrimidine biosynthesis.

108. The method of any one of claims 104-107, wherein the purine nucleotide and/or the purine nucleoside induces cardiac cell death.

109. The method of any one of claims 104-108, wherein the pyrimidine nucleotide is involved in pyrimidine biosynthesis.

110. The method of any one of claims 104-109, wherein the pyrimidine nucleotide prevents cardiac cell death.

111. The method of any one of claims 104-110, wherein the purine nucleotide is adenine.

112. The method of any one of claims 104-110, wherein the purine nucleoside is adenosine.

113. The method of any one of claims 104-110, wherein the purine nucleoside is adenosine monophosphate (AMP).

114. The method of any one of claims 104-110, wherein the purine nucleoside is inosine.

115. The method of any one of claims 104-110, wherein the purine nucleoside is inosine monophosphate (IMP).

116. The method of any one of claims 104-115, wherein the pyrimidine nucleotide is uridine.

117. The method of any one of claims 104-115, wherein the pyrimidine nucleotide is uridine monophosphate (UMP).

118. The method of any one of claims 104-115, wherein the pyrimidine nucleotide is uridine triphosphate (UTP).

119. The method of any one of claims 104-115, wherein the pyrimidine nucleotide is cytidine.

120. The method of any one of claims 104-115, wherein the pyrimidine nucleotide is cytidine monophosphate (CMP).

121. The method of any one of claims 104-115, wherein the pyrimidine nucleotide is cytidine triphosphate (CTP).

122. The method of any one of claims 104-115, wherein the pyrimidine nucleotide is orotate.

123. The method of any one of claims 104-115, wherein the pyrimidine nucleotide is deoxyuridine.

124. The method of any one of claims 104-115, wherein the pyrimidine nucleotide is orotidine.

125. The method of any one of claims 104-122, further comprising measuring a level of cell death in the cell sample contacted with the test agent and determining if the level of cell death is decreased as compared to a level of cell death of the cell sample not contacted with the test agent.
